(19) United States
(12) Patent Application Publication
(10) Pub. No.: US 2004/0209956 A1
(43) Pub. Date: Oct. 21, 2004

(54) S-[2-[(1-Iminoethyl)amino]ethyl]-2-methyl-L-cysteine maleate form II crystalline salt

(76) Inventors: Lyle Brostrom, Lincolnshire, IL (US); Ahmad Sheikh, Dearfield, IL (US)

Correspondence Address: Pharmacia Corporation, Corporate Patent Department, P.O. Box 1027, Chesterfield, MO, 63006, US

(21) Appl. No.: 10/797,500

(22) Filed: Mar. 10, 2004

Related U.S. Application Data
(60) Provisional application No. 60/453,782, filed on Mar. 11, 2003.

(51) Int. Cl. A61K 31/198

(52) U.S. Cl. 514/562; 562/556

(57) ABSTRACT

Form II crystalline maleate salt of S-[2-[(1-Iminoethyl)amino]ethyl]-2-methyl-L-cysteine is disclosed. The Form II crystalline salt is a channel hydrate, with a melting point of about 77.69° C.

Priority is claimed from U.S. Provisional Application Serial No. 60/453,782, filed Mar. 11, 2003 incorporated herein by reference

### FIELD OF THE INVENTION

The present invention comprises a novel compound useful in the treatment of disease, and more particularly a novel salt of S-[2-[(1-Iminoethyl)amino]ethyl]-2-methyl-L-cysteine, and even more particularly a novel crystalline state (Form II) of a crystalline S-[2-[(1-Iminoethyl)amino]ethyl]-2-methyl-L-cysteine maleate, and pharmaceutical compositions thereof, for the treatment of conditions involving an inappropriate expression of nitric oxide from the inducible isoform of nitric oxide synthase.

S-[2-[(1-Iminoethyl)amino]ethyl]-2-methyl-L-cysteine is described and claimed in commonly assigned U.S. Pat. No. 6,403,830, herein incorporated by reference.

### BACKGROUND OF THE INVENTION

Nitric oxide (NO) is a bioactive free radical gas produced by any one of several isoforms of the enzyme nitric oxide synthase (NOS). The physiological activity of what was later identified as NO was initially discovered in the early 1980's when it was found that vascular relaxation caused by acetylcholine is dependent on the presence of the vascular endothelium. The factor derived from the endothelium, then called endothelium-derived relaxing factor (EDRF), that mediates such vascular relaxation is now known to be NO that is generated in the vascular endothelium by one isoform of NOS. The activity of NO as a vasodilator has been known for well over 100 years. In addition, NO is the active species derived from known nitrovasodilators including amylnitrite, and glyceryltrinitrate. Nitric oxide is also an endogenous stimulator of soluble guanylate cyclase (cGMP), and thus stimulates cGMP production. When NOS is inhibited by N-monomethylarginine (L-NMMA), cGMP formation is completely prevented. In addition to endothelium-dependent relaxation, NO is known to be involved in a number of biological actions including cytotoxicity of phagocytic cells and cell-to-cell communication in the central nervous system.

The identification of EDRF as NO coincided with the discovery of a biochemical pathway by which NO is synthesized from the amino acid L-arginine by the enzyme NO synthase. There are at least three types of NO synthase as follows:

(i) a constitutive, Ca++/calmodulin dependent enzyme, located in the brain, that releases NO in response to receptor or physical stimulation;

(ii) a Ca++ independent enzyme, a 130 kD protein, which is induced after activation of vascular smooth muscle, macrophages, endothelial cells, and a number of other cells by endotoxin and cytokines; and

(iii) a constitutive, Ca++/calmodulin dependent enzyme, located in the endothelium, that releases NO in response to receptor or physical stimulation.

Once expressed, inducible nitric oxide synthase (hereinafter “iNOS”) generates NO continuously for long periods. Clinical studies have shown that NO production and iNOS expression are increased in a variety of chronic inflammatory diseases, such as rheumatoid and osteoarthritis (see, e.g, McInnes I. B. et al., J. Exp. Med. 184:1519 (1996)), inflammatory bowel disease (see, e.g, Lundberg J. O. N. et al., Lancet 344:1673, (1994)), and asthma (see, e.g., Hamid, Q. et al., Lancet 342:1510 (1993)), and iNOS is implicated as a major pathological factor in these chronic inflammatory diseases.

Thus, inhibition of excessive NO production by iNOS is likely to be anti-inflammatory. However, since the production of NO from eNOS and nNOS is involved in normal physiology, it would be desirable for any NOS inhibitor that is used for treating inflammation be selective for iNOS, so that normal physiological modulation of blood pressure by eNOS-generated NO, and non-adrenergic, non-cholinergic neuronal transmission by nNOS-generated NO would remain unaffected.

With all pharmaceutical compounds and compositions, the chemical and physical stability of a drug compound is important in the commercial development of that drug substance. Such stability includes the stability at ambient conditions, especially to moisture and under storage conditions. Elevated stability at different conditions of storage is needed to predict the different possible storage conditions during the lifetime of a commercial product. A stable drug avoids the use of special storage conditions as well as frequent inventory replacement. A drug compound must also be stable during the manufacturing process which often requires milling of the drug to achieve drug material with uniform particle size and surface area. Unstable materials often undergo polymorphic changes. Therefore, any modification of a drug substance which enhances its stability profile provides a meaningful benefit over less stable substances.

Several inhibitors of iNOS have been described, such as, for example, S-[2-[(1-Iminoethyl)amino]ethyl]-2-methyl-L-cysteine, which is described and claimed in commonly assigned U.S. Pat. No. 6,403,830. That compound, however, is an amorphous solid. It would be desirable, therefore, to provide a crystalline solid form of an iNOS inhibitor such as S-[2-[(1-Iminoethyl)amino]ethyl]-2-methyl-L-cysteine.

### BRIEF DESCRIPTION OF THE DRAWINGS

FIG. 1 is a schematic of S-[2-[(1-Iminoethyl)amino]ethyl]-2-methyl-L-cysteine titration curve, showing all ionization states;

FIG. 2 is a graphical representation of titration curves of S-[2-[(1-Iminoethyl)amino]ethyl]-2-methyl-L-cysteine in water with IRA-400(OH) anion exchange resin. Diamond is pH and square (dashed line) is S-[2-[(1-Iminoethyl)amino]ethyl]-2-methyl-L-cysteine (% initial, by ion chromatography);

FIG. 3 is a graphical representation of titration curves of S-[2-[(1-Iminoethyl)amino]ethyl]-2-methyl-L-cysteine in water with IRA-400(OH) anion exchange resin. Diamond is pH and triangle (broken line) is chloride (by ion chromatography);

FIG. 4 Shows titration curves of S-[2-[(1-Iminoethyl)amino]ethyl]-2-methyl-L-cysteine in water with IRA-400 anion exchange resin;

FIG. 5 shows the relevant binding data associated with increasing pH of the zwitterion of S-[2-[(1-Iminoethyl)amino]ethyl]-2-methyl-L-cysteine;

FIG. 6 is a powder x-ray pattern of a sample (Example 12) of S-[2-[(1-Iminoethyl)amino]ethyl]-2-methyl-L-cysteine maleate (Form I);

FIG. 7 is a powder x-ray pattern of a sample (Example 11) of S-[2-[(1-Iminoethyl)amino]ethyl]-2-methyl-L-cysteine maleate (Form I);

FIG. 8 is a graph of a differential scanning calorimetry study of a sample (10.046 mg. Example 12) of S-[2-[(1-Iminoethyl)amino]ethyl]-2-methyl-L-cysteine maleate (Form I);

FIG. 9 is a graph of a differential scanning calorimetry study of a sample (6.2130 mg. Example 11) of S-[2-[(1-Iminoethyl)amino]ethyl]-2-methyl-L-cysteine maleate (Form I);

FIG. 10 is a thermogravimetric plot of a sample (4.7680 mg. Example 12) of S-[2-[(1-Iminoethyl)amino]ethyl]-2-methyl-L-cysteine maleate (Form I);

FIG. 11 is a thermogravimetric plot of a sample (Example 11) of S-[2-[(1-Iminoethyl)amino]ethyl]-2-methyl-L-cysteine maleate (Form I);

FIG. 12 is a plot of a moisture sorption study of S-[2-[(1-Iminoethyl)amino]ethyl]-2-methyl-L-cysteine maleate (Form I);

FIG. 13 is the Raman spectrum of a sample of S-[2-[(1-Iminoethyl)amino]ethyl]-2-methyl-L-cysteine maleate (Form I);

FIG. 14 is a representation of a unit cell of S-[2-[(1-Iminoethyl)amino]ethyl]-2-methyl-L-cysteine maleate (Form II);

FIG. 15 is a powder x-ray pattern of a sample of S-[2-[(1-Iminoethyl)amino]ethyl]-2-methyl-L-cysteine maleate (Form II);

FIG. 16 is a comparison of simulated and observed powder x-ray pattern of a sample of S-[2-[(1-Iminoethyl)amino]ethyl]-2-methyl-L-cysteine maleate (Form II);

FIG. 17 is graph of a differential scanning calorimetry study of a sample of S-[2-[(1-Iminoethyl)amino]ethyl]-2-methyl-L-cysteine maleate (Form II);

FIG. 18 is a thermogravimetric plot of a sample (3.7520 mg.) of S-[2-[(1-Iminoethyl)amino]ethyl]-2-methyl-L-cysteine maleate (Form II); and

FIG. 19 is a plot of a moisture sorption study of S-[2-[(1-Iminoethyl)amino]ethyl]-2-methyl-L-cysteine maleate (Form II).

### SUMMARY OF THE INVENTION

The present invention is directed to a novel crystalline salt form of S-[2-[(1-Iminoethyl)amino]ethyl]-2-methyl-L-cysteine, pharmaceutical compositions, a process for preparing the novel salt compounds, a process for preparing pharmaceutical compositions, and methods of using said novel Form II crystalline salt compound and compositions for inhibiting or modulating nitric oxide synthesis in a subject in need of such inhibition or modulation by administering a salt of a compound which preferentially inhibits or modulates the inducible isoform of nitric oxide synthase over the constitutive isoforms of nitric oxide synthase. The present salt compound possesses useful nitric oxide synthase inhibiting activity, and is expected to be useful in the treatment or prophylaxis of a disease or condition in which the synthesis or oversynthesis of nitric oxide forms a contributory part.

Stoichiometrically, a unit cell of the novel salt is one molecule of S-[2-[(1-Iminoethyl)amino]ethyl]-2-methyl-L-cysteine and one molecule of maleic acid.

The novel crystalline Form II salt is characterized by some or all of the following physical measurements: elemental analysis (such as by combustion analysis), melting point and heat of fusion (differential scanning calorimetry and thermogravimetric analysis), refractive indices (polarized light microscopy), x-ray powder diffraction pattern, and moisture sorption (for example, DVS moisture balance).

The present novel salt can be used to treat diseases involving cartilage degeneration, which takes place in certain conditions such as arthritis. Accordingly, conditions in which there is an advantage in inhibiting NO production from L-arginine include arthritic conditions such as rheumatoid arthritis, osteoarthritis, gouty arthritis, juvenile arthritis, septic arthritis, spondyloarthritis, acute rheumatic arthritis, enteropathic arthritis, neuropathic arthritis, and pyogenic arthritis. In addition, NO-induced depression of chondrocyte respiration could modulate matrix loss and secondary cartilage mineralization in arthritis, in particular osteoarthritis.

Other conditions for which the present salt may be useful include chronic or inflammatory bowel disease, cardiovascular ischemia, diabetes, congestive heart failure, myocarditis, atherosclerosis, migraine, glaucoma, aortic aneurysm, reflux esophagitis, diarrhea, irritable bowel syndrome, cystic fibrosis, emphysema, asthma, bronchiectasis, hyperalgesia, cerebral ischemia, thrombotic stroke, global ischemia (secondary to cardiac arrest), multiple sclerosis and other central nervous system disorders mediated by NO, for example Parkinson's disease and Alzheimer's disease. Further neurodegenerative disorders in which NO inhibition may be useful include nerve degeneration and/or nerve necrosis in disorders such as hypoxia, hypoglycemia, epilepsy, and in external wounds (such as spinal cord and head injury), hyperbaric oxygen convulsions and toxicity, dementia e.g. pre-senile dementia, and AIDS-related dementia, Sydenham's chorea, Huntington's disease, Amyotrophic Lateral Sclerosis, Korsakoffs disease, imbecility relating to a cerebral vessel disorder, sleeping disorders, schizophrenia, depression, depression or other symptoms associated with Premenstrual Syndrome (PMS), anxiety and septic shock.

The present salt may also be used where nitric oxide inhibition may also play a role in the treatment, such as pain including somatogenic (either nociceptive or neuropathic), both acute and chronic. The present compounds could be used in any situation that a common NSAID or opioid analgesic would traditionally be administered.

Still, other disorders that may be treated by inhibiting NO production with the present salt include opiate tolerance in patients needing protracted opiate analgesics, and benzodiazepine tolerance in patients taking benzodiazepines, and other addictive behavior, for example, nicotine and eating disorders. The present compounds may also be useful as antibacterial agents.

Further conditions in which the present salt may be used to inhibit NO production from L-arginine include systemic hypotension associated with septic and/or toxic shock induced by a wide variety of agents; therapy with cytokines such as TNF, IL-1 and IL-2; and as an adjuvant to short term immunosuppression in transplant therapy.

The present salt may also be useful in the treatment of ocular conditions (such as ocular hypertension retinitis uveitis), systemic lupus erythematosis (SLE), glomerulonephritis, restenosis, inflammatory sequelae of viral infections, acute respiratory distress syndrome (ARDS), oxidant-induced lung injury, IL2 therapy such as in a cancer patient, cachexia, immunosuppression such as in transplant therapy, disorders of gastrointestinal motility, sunburn, eczema, psoriasis, gingivitis, pancreatitis, damage to the gastrointestinal tract resulting from infections, cystic fibrosis, treatment to a dysfunctional immune system such as an adjuvant to short term immunosuppression in organ transplant therapy, induction of labor, adenomatous polyposis, controlling tumor growth, chemotherapy, chemoprevention and bronchitis.

The present invention is also directed to pharmaceutical compositions for the treatment of pain, asthma and other airway disorders, cancer, arthritis, ocular disorders including retinopathies and glaucoma, inflammation related disorders including irritable bowel syndrome, and other disorders in which an excessive production of nitric oxide plays a role, which comprises a therapeutically effective amount of crystalline S-[2-[(1-Iminoethyl)amino]ethyl]-2-methyl-L-cysteine maleate Form II together with a pharmaceutically acceptable carrier, diluent or vehicle.

Besides being useful for human treatment, this form is also useful for veterinary treatment of companion animals, exotic animals and farm animals, including mammals, rodents, and the like. More preferred animals include horses, dogs, and cats.

### DETAILED DESCRIPTION OF THE INVENTION

### Definitions

The terms “treat,” “treating” and “treatment,” as used herein includes prophylactic, palliative treatment, or restorative treatment.

The term “effective amount” means a dose conducive to treatment. An effective amount may be administered in a single dose, or in divided doses over a period of time.

The term “ACE” means acetone.

The term “ACN” means acetonitrile.

The term “amorphous” as applied to S-[2-[(1-Iminoethyl)amino]ethyl]-2-methyl-L-cysteine herein refers to a solid state wherein the S-[2-[(1-Iminoethyl)amino]ethyl]-2-methyl-L-cysteine molecules are present in a disordered arrangement and do not form a distinguishable crystal lattice or unit cell. When subjected to X-ray powder diffraction, amorphous S-[2-[(1-Iminoethyl)amino]ethyl]-2-methyl-L-cysteine does not produce any characteristic crystalline peaks.

The term “crystalline form” as applied to S-[2-[(1-Iminoethyl)amino]ethyl]-2-methyl-L-cysteine herein refers to a solid state form wherein the S-[2-[(1-Iminoethyl)amino]ethyl]-2-methyl-L-cysteine molecules are arranged to form a distinguishable crystal lattice (i) comprising distinguishable unit cells, and (ii) yielding diffraction peaks when subjected to X-ray radiation.

The terms “S-[2-[(1-Iminoethyl)amino]ethyl]-2-methyl-L-cysteine maleate Form I,” S-[2-[(1-Iminoethyl)amino]ethyl]-2-methyl-L-cysteine Form I” and “Form I” all mean S-[2-[(1-Iminoethyl)amino]ethyl]-2-methyl-L-cysteine maleate crystalline salt Form I, as more fully described herein.

The terms “S-[2-[(1-Iminoethyl)amino]ethyl]-2-methyl-L-cysteine maleate Form II,” S-[2-[(1-Iminoethyl)amino]ethyl]-2-methyl-L-cysteine Form II” and “Form II” all mean S-[2-[(1-Iminoethyl)amino]ethyl]-2-methyl-L-cysteine maleate crystalline salt Form II, as more fully described herein.

The term “crystallization” as used herein can refer to crystallization and/or recrystallization depending upon the applicable circumstances relating to preparation of S-[2-[(1-Iminoethyl)amino]ethyl]-2-methyl-L-cysteine starting material.

The term “DMF” means N,N-dimethylformamide.

The term “D/W/A” refers to a ternary solvent system of N,N-dimethylformamide (DMF), water and acetonitrile.

The term “S-[2-[(1-Iminoethyl)amino]ethyl]-2-methyl-L-cysteine drug substance” as used herein means S-[2-[(1-Iminoethyl)amino]ethyl]-2-methyl-L-cysteine per se as qualified by the context in which the term is used, and can refer to unformulated S-[2-[(1-Iminoethyl)amino]ethyl]-2-methyl-L-cysteine or to S-[2-[(1-Iminoethyl)amino]ethyl]-2-methyl-L-cysteine present as an ingredient of a pharmaceutical composition.

The term “DSC” means differential scanning calorimetry.

The term “HPLC” means high pressure liquid chromatography.

The term “IR” means infrared.

The term “NMR” means nuclear magnetic resonance, and may apply to nuclear magnetic resonance spectroscopy.

The term “ml” means milliliters.

The term “mg” means milligrams.

The term “μg” means micrograms

The term “μl” means microliters.

The term “nucleation,” as used herein, means the formation of crystals in a solution.

The term “Purity” herein, unless otherwise qualified, means the chemical purity of S-[2-[(1-Iminoethyl)amino]ethyl]-2-methyl-L-cysteine according to conventional HPLC assay.

The term “PXRD” means powder X-ray diffraction.

The term “rpm” means revolutions per minute.

The term “seeding,” as used herein, means the addition of crystals to a solution for the purpose of initiating or enhancing nucleation.

The term “TGA” means thermogravimetric analysis.

The term “Tm” means melting temperature.

The term “free zwitterion” means a molecule that carries both a positive and negative charge such that the net charge is zero.

### Pharmaceutical Use

S-[2-[(1-Iminoethyl)amino]ethyl]-2-methyl-L-cysteine maleate crystalline salt Form II will be useful for treating, among other things, inflammation in a subject, or for treating other nitric oxide synthase-mediated disorders, such as, as an analgesic in the treatment of pain and headaches, or as an antipyretic for the treatment of fever. For example, S-[2-[(1-Iminoethyl)amino]ethyl]-2-methyl-L-cysteine maleate crystalline salt Form II will be useful to treat arthritis, including but not limited to rheumatoid arthritis, spondyloarthropathies, gouty arthritis, osteoarthritis, systemic lupus erythematosus, juvenile arthritis, acute rheumatic arthritis, enteropathic arthritis, neuropathic arthritis, psoriatic arthritis, and pyogenic arthritis. Conditions in which the S-[2-[(1-Iminoethyl)amino]ethyl]-2-methyl-L-cysteine maleate crystalline salt Form II will provide an advantage in inhibiting NO production from L-arginine include arthritic conditions.

S-[2-[(1-Iminoethyl)amino]ethyl]-2-methyl-L-cysteine maleate crystalline salt Form II will be further useful in the treatment of asthma, bronchitis, menstrual cramps (e.g., dysmenorrhea), premature labor, tendinitis, bursitis, skin-related conditions such as psoriasis, eczema, bums, sunburn, dermatitis, pancreatitis, hepatitis, and from post-operative inflammation including from ophthalmic surgery such as cataract surgery and refractive surgery. S-[2-[(1-Iminoethyl)amino]ethyl]-2-methyl-L-cysteine maleate crystalline salt Form II also would be useful to treat gastrointestinal conditions such as inflammatory bowel disease, Crohn's disease, gastritis, irritable bowel syndrome and ulcerative colitis.

S-[2-[(1-Iminoethyl)amino]ethyl]-2-methyl-L-cysteine maleate crystalline salt Form II would be useful for the prevention or treatment of cancer, such as colorectal cancer, and cancer of the breast, lung, prostate, bladder, cervix and skin. S-[2-[(1-Iminoethyl)amino]ethyl]-2-methyl-L-cysteine maleate crystalline salt Form II of the invention would be useful in treating inflammation and tissue damage in such diseases as vascular diseases, migraine headaches, periarteritis nodosa, thyroiditis, aplastic anemia, Hodgkin's disease, scierodoma, rheumatic fever, type I diabetes, neuromuscular junction disease including myasthenia gravis, white matter disease including multiple sclerosis, sarcoidosis, nephrotic syndrome, Behcet's syndrome, polymyositis, gingivitis, nephritis, hypersensitivity, swelling occurring after injury, myocardial ischemia, and the like. The S-[2-[(1-Iminoethyl)amino]ethyl]-2-methyl-L-cysteine maleate crystalline salt Form II would also be useful in the treatment of ophthalmic diseases, such as glaucoma, retinitis, retinopathies, uveitis, ocular photophobia, and of inflammation and pain associated with acute injury to the eye tissue. Of particular interest among the uses of the present inventive S-[2-[(1-Iminoethyl)amino]ethyl]-2-methyl-L-cysteine maleate crystalline salt Form II is the treatment of glaucoma, especially where symptoms of glaucoma are caused by the production of nitric oxide, such as in nitric oxide-mediated nerve damage. The S-[2-[(1-Iminoethyl)amino]ethyl]-2-methyl-L-cysteine maleate crystalline salt Form II would also be useful in the treatment of pulmonary inflammation, such as that associated with viral infections and cystic fibrosis. The S-[2-[(1-Iminoethyl)amino]ethyl]-2-methyl-L-cysteine maleate crystalline salt Form II would also be useful for the treatment of certain central nervous system disorders, such as cortical dementias including Alzheimer's disease, and central nervous system damage resulting from stroke, ischemia and trauma. The S-[2-[(1-Iminoethyl)amino]ethyl]-2-methyl-L-cysteine maleate crystalline salt Form II is useful as an anti-inflammatory agent, such as for the treatment of arthritis, with the additional benefit of having significantly less harmful side effects.

S-[2-[(1-Iminoethyl)amino]ethyl]-2-methyl-L-cysteine maleate crystalline salt Form II would also be useful in the treatment of allergic rhinitis, respiratory distress syndrome, endotoxin shock syndrome, and atherosclerosis.

S-[2-[(1-Iminoethyl)amino]ethyl]-2-methyl-L-cysteine maleate crystalline salt Form II would also be useful in the treatment of pain, including but not limited to postoperative pain, dental pain, muscular pain, and pain resulting from cancer. S-[2-[(1-Iminoethyl)amino]ethyl]-2-methyl-L-cysteine maleate crystalline salt Form II would be useful for the prevention of dementias, such as Alzheimer's disease.

Besides being useful for human treatment, S-[2-[(1-Iminoethyl)amino]ethyl]-2-methyl-L-cysteine maleate crystalline salt Form II is also useful for veterinary treatment of companion animals, exotic animals and farm animals, including mammals, rodents, and the like. More preferred animals include horses, dogs, and cats.

The present S-[2-[(1-Iminoethyl)amino]ethyl]-2-methyl-L-cysteine maleate crystalline salt Form II may also be used in co-therapies, partially or completely, in place of other conventional antiinflammatory therapies, such as together with steroids, NSAIDs, COX-2 selective inhibitors, 5-lipoxygenase inhibitors, LTB₄ antagonists and LTA₄ hydrolase inhibitors.

Other conditions in which the S-[2-[(1-Iminoethyl)amino]ethyl]-2-methyl-L-cysteine maleate crystalline salt Form II of the present invention will provide an advantage in inhibiting NO inhibition include cardiovascular ischemia, diabetes (type I or type II), congestive heart failure, myocarditis, atherosclerosis, migraine, glaucoma, aortic aneurysm, reflux esophagitis, diarrhea, irritable bowel syndrome, cystic fibrosis, emphysema, asthma, bronchiectasis, hyperalgesia (allodynia), cerebral ischemia (both focal ischemia, thrombotic stroke and global ischemia (for example, secondary to cardiac arrest), multiple sclerosis and other central nervous system disorders mediated by NO, for example Parkinson's disease. Further neurodegenerative disorders in which NO inhibition may be useful include nerve degeneration or nerve necrosis in disorders such as hypoxia, hypoglycemia, epilepsy, and in cases of central nervous system (CNS) trauma (such as spinal cord and head injury), hyperbaric oxygen convulsions and toxicity, dementia e.g. pre-senile dementia, and AIDS-related dementia, cachexia, Sydenham's chorea, Huntington's disease, Amyotrophic Lateral Sclerosis, Korsakoffs disease, imbecility relating to a cerebral vessel disorder, sleeping disorders, schizophrenia, depression, depression or other symptoms associated with Premenstrual Syndrome (PMS), anxiety and septic shock.

The S-[2-[(1-Iminoethyl)amino]ethyl]-2-methyl-L-cysteine maleate crystalline salt Form II of the present invention will also be useful in the treatment of pain including somatogenic (either nociceptive or neuropathic), both acute and chronic. S-[2-[(1-Iminoethyl)amino]ethyl]-2-methyl-L-cysteine maleate crystalline salt Form II could be used in any situation including neuropathic pain that a common NSAID or opioid analgesic would traditionally be administered.

Still other disorders or conditions which will be advantageously treated by the S-[2-[(1-Iminoethyl)amino]ethyl]-2-methyl-L-cysteine maleate crystalline salt Form II of the present invention include treatment or prevention of opiate tolerance in patients needing protracted opiate analgesics, and benzodiazepine tolerance in patients taking benzodiazepines, and other addictive behavior, for example, nicotine addiction, alcoholism, and eating disorders.

The S-[2-[(1-Iminoethyl)amino]ethyl]-2-methyl-L-cysteine maleate crystalline salt Form II of the present invention will also be useful in the treatment or prevention of drug withdrawal symptoms, for example treatment or prevention of symptoms of withdrawal from opiate, alcohol, or tobacco addiction.

The S-[2-[(1-Iminoethyl)amino]ethyl]-2-methyl-L-cysteine maleate crystalline salt Form II may also be useful to prevent tissue damage when therapeutically combined with antibacterial or antiviral agents.

The S-[2-[(1-Iminoethyl)amino]ethyl]-2-methyl-L-cysteine maleate crystalline salt Form II of the present invention will also be useful in inhibiting NO production from L-arginine including systemic hypotension associated with septic and/or toxic hemorrhagic shock induced by a wide variety of agents; therapy with cytokines such as TNF, IL-1 and IL-2; and as an adjuvant to short term immunosuppression in transplant therapy.

The present invention is further directed to the use of the S-[2-[(1-Iminoethyl)amino]ethyl]-2-methyl-L-cysteine maleate crystalline salt Form II of the present invention for the treatment and prevention of neoplasias. The neoplasias that will be treatable or preventable by the S-[2-[(1-Iminoethyl)amino]ethyl]-2-methyl-L-cysteine maleate crystalline salt Form II and methods of the present invention include brain cancer, bone cancer, a leukemia, a lymphoma, epithelial cell-derived neoplasia (epithelial carcinoma) such as basal cell carcinoma, adenocarcinoma, gastrointestinal cancer such as lip cancer, mouth cancer, esophogeal cancer, small bowel cancer and stomach cancer, colon cancer, liver cancer, bladder cancer, pancreas cancer, ovary cancer, cervical cancer, lung cancer, breast cancer and skin cancer, such as squamous cell and basal cell cancers, prostate cancer, renal cell carcinoma, and other known cancers that effect epithelial cells throughout the body. Preferably, the neoplasia to be treated is selected from gastrointestinal cancer, liver cancer, bladder cancer, pancreas cancer, ovary cancer, prostate cancer, cervical cancer, lung cancer, breast cancer and skin cancer, such as squamous cell and basal cell cancers. The present S-[2-[(1-Iminoethyl)amino]ethyl]-2-methyl-L-cysteine maleate crystalline salt Form II and methods can also be used to treat the fibrosis which occurs with radiation therapy. The present S-[2-[(1-Iminoethyl)amino]ethyl]-2-methyl-L-cysteine maleate crystalline salt Form II and methods can be used to treat subjects having adenomatous polyps, including those with familial adenomatous polyposis (FAP). Additionally, the present S-[2-[(1-Iminoethyl)amino]ethyl]-2-methyl-L-cysteine maleate crystalline salt Form II and methods can be used to prevent polyps from forming in patients at risk of FAP.

Conjunctive treatment of a S-[2-[(1-Iminoethyl)amino]ethyl]-2-methyl-L-cysteine maleate crystalline salt Form II of the present invention with another antineoplastic agent will produce a synergistic effect or alternatively reduce the toxic side effects associated with chemotherapy by reducing the therapeutic dose of the side effect-causing agent needed for therapeutic efficacy or by directly reducing symptoms of toxic side effects caused by the side effect-causing agent. S-[2-[(1-Iminoethyl)amino]ethyl]-2-methyl-L-cysteine maleate crystalline salt Form II of the present invention will further be useful as an adjunct to radiation therapy to reduce side effects or enhance efficacy.

In the present invention, another agent which can be combined therapeutically with the S-[2-[(1-Iminoethyl)amino]ethyl]-2-methyl-L-cysteine maleate crystalline salt Form II of the present invention includes any therapeutic agent which is capable of inhibiting the enzyme cyclooxygenase-2 (“COX-2”). Preferably such COX-2 inhibiting agents inhibit COX-2 selectively relative to the enzyme cyclooxygenase-1 (“COX-1”). Such a COX-2 inhibitor is known as a “COX-2 selective inhibitor”. COX-2 selective inhibitors useful in therapeutic combination with the S-[2-[(1-Iminoethyl)amino]ethyl]-2-methyl-L-cysteine maleate crystalline salt Form II of the present invention include celecoxib, valdecoxib, deracoxib, etoricoxib, rofecoxib, ABT-963 (2-(3,4-difluorophenyl)-4-(3-hydroxy-3-methyl-1-butoxy)-5-[4-(methylsulfonyl)phenyl-3(2H)-pyridazinone; described in PCT Patent Application No. WO 00/24719), or meloxicam. S-[2-[(1-Iminoethyl)amino]ethyl]-2-methyl-L-cysteine maleate crystalline salt Form II of the present invention can also be advantageously used in therapeutic combination with a prodrug of a COX-2 selective inhibitor, for example parecoxib.

Another chemotherapeutic agent which will be useful in combination with the S-[2-[(1-Iminoethyl)amino]ethyl]-2-methyl-L-cysteine maleate crystalline salt Form II of the present invention can be selected, for example, from the following non-comprehensive and non-limiting list:

Alpha-difluoromethylornithine (DFMO), 5-FU-fibrinogen, acanthifolic acid, aminothiadiazole, brequinar sodium, carmofur, Ciba-Geigy CGP-30694, cyclopentyl cytosine, cytarabine phosphate stearate, cytarabine conjugates, Lilly DATHF, Merrel Dow DDFC, dezaguanine, dideoxycytidine, dideoxyguanosine, didox, Yoshitomi DMDC, doxifluridine, Wellcome EHNA, Merck & Co. EX-015, fazarabine, floxuridine, fludarabine phosphate, 5-fluorouracil, N-(2′-furanidyl)-5-fluorouracil, Daiichi Seiyaku FO-152, isopropyl pyrrolizine, Lilly LY-188011, Lilly LY-264618, methobenzaprim, methotrexate, Wellcome MZPES, norspermidine, NCI NSC-127716, NCI NSC-264880, NCI NSC-39661, NCI NSC-612567, Warner-Lambert PALA, pentostatin, piritrexim, plicamycin, Asahi Chemical PL-AC, Takeda TAC-788, thioguanine, tiazofurin, Erbamont TIF, trimetrexate, tyrosine kinase inhibitors, tyrosine protein kinase inhibitors, Taiho UFT, uricytin, Shionogi 254-S, aldo-phosphamide analogues, altretamine, anaxirone, Boehringer Mannheim BBR-2207, bestrabucil, budotitane, Wakunaga CA-102, carboplatin, carmustine, Chinoin-139, Chinoin-153, chlorambucil, cisplatin, cyclophosphamide, American Cyanamid CL-286558, Sanofi CY-233, cyplatate, Degussa D-19-384, Sumimoto DACHP(Myr)2, diphenylspiromustine, diplatinum cytostatic, Erba distamycin derivatives, Chugai DWA-2114R, ITI E09, elmustine, Erbamont FCE-24517, estramustine phosphate sodium, fotemustine, Unimed G-6-M, Chinoin GYKI-17230, hepsul-fam, ifosfamide, iproplatin, lomustine, mafosfamide, mitolactol, Nippon Kayaku NK-121, NCI NSC-264395, NCI NSC-342215, oxaliplatin, Upjohn PCNU, prednimustine, Proter PTT-119, ranimustine, semustine, SmithKline SK&F-101772, Yakult Honsha SN-22, spiromus-tine, Tanabe Seiyaku TA-077, tauromustine, temozolomide, teroxirone, tetraplatin, trimelamol, Taiho 4181-A, aclarubicin, actinomycin D, actinoplanone, Erbamont ADR-456, aeroplysinin derivative, Ajinomoto AN-201-II, Ajinomoto AN-3, Nippon Soda anisomycins, anthracycline, azino-mycin-A, bisucaberin, Bristol-Myers BL-6859, Bristol-Myers BMY-25067, Bristol-Myers BMY-25551, Bristol-Myers BMY-26605, Bristol-Myers BMY-27557, Bristol-Myers BMY-28438, bleomycin sulfate, bryostatin-1, Taiho C-1027, calichemycin, chromoximycin, dactinomycin, daunorubicin, Kyowa Hakko DC-102, Kyowa Hakko DC-79, Kyowa Hakko DC-88A, Kyowa Hakko DC89-A1, Kyowa Hakko DC92-B, ditrisarubicin B, Shionogi DOB-41, doxorubicin, doxorubicin-fibrinogen, elsamicin-A, epirubicin, erbstatin, esorubicin, esperamicin-A1, esperamicin-A1b, Erbamont FCE-21954, Fujisawa FK-973, fostriecin, Fujisawa FR-900482, glidobactin, gregatin-A, grincamycin, herbimycin, idarubicin, illudins, kazusamycin, kesarirhodins, Kyowa Hakko KM-5539, Kirin Brewery KRN-8602, Kyowa Hakko KT-5432, Kyowa Hakko KT-5594, Kyowa Hakko KT-6149, American Cyanamid LL-D49194, Meiji Seika ME 2303, menogaril, mitomycin, mitoxantrone, SmithKline M-TAG, neoenactin, Nippon Kayaku NK-313, Nippon Kayaku NKT-01, SRI International NSC-357704, oxalysine, oxaunomycin, peplomycin, pilatin, pirarubicin, porothramycin, pyrindamycin A, Tobishi RA-I, rapamycin, rhizoxin, rodorubicin, sibanomicin, siwenmycin, Sumitomo SM-5887, Snow Brand SN-706, Snow Brand SN-07, sorangicin-A, sparsomycin, SS Pharmaceutical SS-21020, SS Pharmaceutical SS-7313B, SS Pharmaceutical SS-9816B, steffimycin B, Taiho 4181-2, talisomycin, Takeda TAN-868A, terpentecin, thrazine, tricrozarin A, Upjohn U-73975, Kyowa Hakko UCN-10028A, Fujisawa WF-3405, Yoshitomi Y-25024 zorubicin, alpha-carotene, alpha-difluoromethyl-arginine, acitretin, Biotec AD-5, Kyorin AHC-52, alstonine, amonafide, amphethinile, amsacrine, Angiostat, ankinomycin, anti-neoplaston A10, antineoplaston A2, antineoplaston A3, antineoplaston A5, antineoplaston AS2-1, Henkel APD, aphidicolin glycinate, asparaginase, Avarol, baccharin, batracylin, benfluron, benzotript, Ipsen-Beaufour BIM-23015, bisantrene, Bristo-Myers BMY-40481, Vestar boron-10, bromofosfamide, Wellcome BW-502, Wellcome BW-773, caracemide, carmethizole hydrochloride, Ajinomoto CDAF, chlorsulfaquinoxalone, Chemex CHX-2053, Chemex CHX-100, Warner-Lambert CI-921, Warner-Lambert CI-937, Warner-Lambert CI-941, Warner-Lambert CI-958, clanfenur, claviridenone, ICN compound 1259, ICN compound 4711, Contracan, Yakult Honsha CPT-11, crisnatol, curaderm, cytochalasin B, cytarabine, cytocytin, Merz D-609, DABIS maleate, dacarbazine, datelliptinium, didemnin-B, dihaematoporphyrin ether, dihydrolenperone, dinaline, distamycin, Toyo Pharmar DM-341, Toyo Pharmar DM-75, Daiichi Seiyaku DN-9693, elliprabin, elliptinium acetate, Tsumura EPMTC, ergotamine, etoposide, etretinate, fenretinide, Fujisawa FR-57704, gallium nitrate, genkwadaphnin, Chugai GLA-43, Glaxo GR-63178, grifolan NMF-5N, hexadecylphosphocholine, Green Cross HO-221, homoharringtonine, hydroxyurea, BTG ICRF-187, ilmofosine, isoglutamine, isotretinoin, Otsuka JI-36, Ramot K-477, Otsuak K-76COONa, Kureha Chemical K-AM, MECT Corp KI-8110, American Cyanamid L-623, leukoregulin, lonidamine, Lundbeck LU-23-112, Lilly LY-186641, NCI (US) MAP, marycin, Merrel Dow MDL-27048, Medco MEDR-340, merbarone, merocyanine derivatives, methylanilinoacridine, Molecular Genetics MGI-136, minactivin, mitonafide, mitoquidone, mopidamol, motretinide, Zenyaku Kogyo MST-16, N-(retinoyl)amino acids, Nisshin Flour Milling N-021, N-acylated-dehydroalanines, nafazatrom, Taisho NCU-190, nocodazole derivative, Normosang, NCI NSC-145813, NCI NSC-361456, NCI NSC-604782, NCI NSC-95580, octreotide, Ono ONO-112, oquizanocine, Akzo Org-10172, pancratistatin, pazelliptine, Warner-Lambert PD-111707, Warner-Lambert PD-115934, Warner-Lambert PD-131141, Pierre Fabre PE-1001, ICRT peptide D, piroxantrone, polyhaematoporphyrin, polypreic acid, Efamol porphyrin, probimane, procarbazine, proglumide, Invitron protease nexin I, Tobishi RA-700, razoxane, Sapporo Breweries RBS, restrictin-P, retelliptine, retinoic acid, Rhone-Poulenc RP-49532, Rhone-Poulenc RP-56976, SmithKline SK&F-104864, Sumitomo SM-108, Kuraray SMANCS, SeaPharm SP-10094, spatol, spirocyclopropane derivatives, spirogermanium, Unimed, SS Pharmaceutical SS-554, strypoldinone, Stypoldione, Suntory SUN 0237, Suntory SUN 2071, superoxide dismutase, Toyama T-506, Toyama T-680, taxol, Teijin TEI-0303, teniposide, thaliblastine, Eastman Kodak TJB-29, tocotrienol, Topostin, Teijin TT-82, Kyowa Hakko UCN-01, Kyowa Hakko UCN-1028, ukrain, Eastman Kodak USB-006, vinblastine sulfate, vincristine, vindesine, vinestramide, vinorelbine, vintriptol, vinzolidine, withanolides, Yamanouchi YM-534, uroguanylin, combretastatin, dolastatin, idarubicin, epirubicin, estramustine, cyclophosphamide, 9-amino-2-(S)-camptothecin, topotecan, irinotecan (Camptosar), exemestane, decapeptyl (tryptorelin), or an omega-3 fatty acid.

Examples of radioprotective agents which may be used in a combination therapy with the S-[2-[(1-Iminoethyl)amino]ethyl]-2-methyl-L-cysteine maleate crystalline salt Form II of this invention include AD-5, adchnon, amifostine analogues, detox, dimesna, 1-102, MM-159, N-acylated-dehydroalanines, TGF- Genentech, tiprotimod, amifostine, WR-151327, FUT-187, ketoprofen transdermal, nabumetone, superoxide dismutase (Chiron) and superoxide dismutase Enzon.

The S-[2-[(1-Iminoethyl)amino]ethyl]-2-methyl-L-cysteine maleate crystalline salt Form II of the present invention will also be useful in treatment or prevention of angiogenesis-related disorders or conditions, for example, tumor growth, metastasis, macular degeneration, and atherosclerosis.

In a further embodiment, the present invention also provides therapeutic combinations for the treatment or prevention of ophthalmic disorders or conditions such as glaucoma. For example the present inventive S-[2-[(1-Iminoethyl)amino]ethyl]-2-methyl-L-cysteine maleate crystalline salt Form II advantageously will be used in therapeutic combination with a drug which reduces the intraocular pressure of patients afflicted with glaucoma. Such intraocular pressure-reducing drugs include without limitation latanoprost, travoprost, bimatoprost, or unoprostol. The therapeutic combination of the S-[2-[(1-Iminoethyl)amino]ethyl]-2-methyl-L-cysteine maleate crystalline salt Form II of the present invention plus an intraocular pressure-reducing drug will be useful because each is believed to achieve its effects by affecting a different mechanism.

In another combination of the present invention, the present inventive S-[2-[(1-Iminoethyl)amino]ethyl]-2-methyl-L-cysteine maleate crystalline salt Form II can be used in therapeutic combination with an antihyperlipidemic or cholesterol-lowering drug such as a benzothiepine or a benzothiazepine antihyperlipidemic drug. Examples of benzothiepine antihyperlipidemic drugs useful in the present inventive therapeutic combination can be found in U.S. Pat. No. 5,994,391, herein incorporated by reference. Some benzothiazepine antihyperlipidemic drugs are described in WO 93/16055. Alternatively, the antihyperlipidemic or cholesterol-lowering drug useful in combination with a compound of the present invention can be an HMG Co-A reductase inhibitor. Examples of HMG Co-A reductase inhibitors useful in the present therapeutic combination include, individually, benfluorex, fluvastatin, lovastatin, provastatin, simvastatin, atorvastatin, cerivastatin, bervastatin, ZD-9720 (described in PCT Patent Application No. WO 97/06802), ZD-4522 (CAS No. 147098-20-2 for the calcium salt; CAS No. 147098-18-8 for the sodium salt; described in European Patent No. EP 521471), BMS 180431 (CAS No. 129829-03-4), or NK-104 (CAS No. 141750-63-2). The therapeutic combination of the S-[2-[(1-Iminoethyl)amino]ethyl]-2-methyl-L-cysteine maleate crystalline salt Form II of the present invention plus an antihyperlipidemic or cholesterol-lowering drug will be useful, for example, in reducing the risk of formation of atherosclerotic lesions in blood vessels. For example, atherosclerotic lesions often initiate at inflamed sites in blood vessels. It is established that antihyperlipidemic or cholesterol-lowering drug reduce risk of formation of atherosclerotic lesions by lowering lipid levels in blood. Without limiting the invention to a single mechanism of action, it is believed that one way the S-[2-[(1-Iminoethyl)amino]ethyl]-2-methyl-L-cysteine maleate crystalline salt Form II of the present combination will work in concert to provide improved control of atherosclerotic lesions by, for example, reducing inflammation of the blood vessels in concert with lowering blood lipid levels.

In another embodiment of the invention, the present S-[2-[(1-Iminoethyl)amino]ethyl]-2-methyl-L-cysteine maleate crystalline salt Form II can be used in combination with other compounds or therapies for the treatment of central nervous conditions or disorders such as migraine. For example, the present S-[2-[(1-Iminoethyl)amino]ethyl]-2-methyl-L-cysteine maleate crystalline salt Form II can be used in therapeutic combination with caffeine, a 5-HT-1B/1D agonist (for example, a triptan such as sumatriptan, naratriptan, zolmitriptan, rizatriptan, almotriptan, or frovatriptan), a dopamine D4 antagonist (e.g., sonepiprazole), aspirin, acetaminophen, ibuprofen, indomethacin, naproxen sodium, isometheptene, dichloralphenazone, butalbital, an ergot alkaloid (e.g., ergotamine, dihydroergotamine, bromocriptine, ergonovine, or methyl ergonovine), a tricyclic antidepressant (e.g., amitriptyline or nortriptyline), a serotonergic antagonist (e.g., methysergide or cyproheptadine), a beta-andrenergic antagonist (e.g., propranolol, timolol, atenolol, nadolol, or metprolol), or a monoamine oxidase inhbitor (e.g., phenelzine or isocarboxazid).

A further embodiment provides a therapeutic combination of the S-[2-[(1-Iminoethyl)amino]ethyl]-2-methyl-L-cysteine maleate crystalline salt Form II of the present invention with an opioid compound. Opioid compounds useful in this combination include without limitation morphine, methadone, hydromorphone, oxymorphone, levorphanol, levallorphan, codeine, dihydrocodeine, dihydrohydroxycodeinone, pentazocine, hydrocodone, oxycodone, nalmefene, etorphine, levorphanol, fentanyl, sufentanil, DAMGO, butorphanol, buprenorphine, naloxone, naltrexone, CTOP, diprenorphine, beta-funaltrexamine, naloxonazine, nalorphine, pentazocine, nalbuphine, naloxone benzoylhydrazone, bremazocine, ethylketocyclazocine, U50,488, U69,593, spiradoline, nor-binaltorphimine, naltrindole, DPDPE, [D-la², glu⁴]deltorphin, DSLET, met-enkephalin, leu-enkaphalin, beta-endorphin, dynorphin A, dynorphin B, and alpha-neoendorphin. An advantage to the combination of the S-[2-[(1-Iminoethyl)amino]ethyl]-2-methyl-L-cysteine maleate crystalline salt Form II of the present invention with an opioid compound is that the present inventive S-[2-[(1-Iminoethyl)amino]ethyl]-2-methyl-L-cysteine maleate crystalline salt Form II will allow a reduction in the dose of the opioid compound, thereby reducing the risk or severity of opioid side effects, such as opioid addiction.

A method to make S-[2-[(1-Iminoethyl)amino]ethyl]-2-methyl-L-cysteine dihydrochloride is described in commonly assigned U.S. Pat. No. 6,403,830, incorporated herein by reference.

Briefly, synthesis of S-[2-[(1-Iminoethyl)amino]ethyl]-2-methyl-L-cysteine dihydrochloride may be performed as in the following Example 1:

### EXAMPLE 1

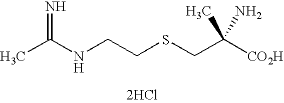

### S-[2-[(1-Iminoethyl)amino]ethyl]-2-methyl-L-cysteine, dihydrochloride

### EXAMPLE-1A

### N-Boc-cysteamine

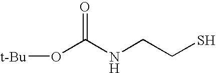

A 3 L 4-neck RB flask was purged with nitrogen for 20 min and then charged sequentially with 2-aminoethanethiol hydrochloride (113.6 g, 1 mol), di-tert-butyl-dicarbonate (218.3 g, 1 mol) and 500 mL of toluene. The mixture was cooled with an ice-water bath and purged with nitrogen for 10 min. Sodium hydroxide (2.5N, 880 mL, 2.2 mol) was added to the stirring mixture in about 1.5 h at between 0 and 11° C. After the addition of sodium hydroxide was complete, the cooling bath was removed and the resulting reaction mixture was allowed to warm up to room temperature and stirred at ambient temperature overnight. This provided a solution of the title product.

### EXAMPLE-1B

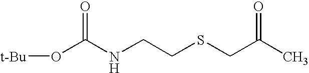

The product solution of Example-1A was cooled with an ice-water bath. A sample of chloroacetone (101.8 g, 1.1 mol) was added to the vigorously stirred reaction mixture over about 50 min at between 8 and 11° C. After the addition of chloroacetone was completed, the cooling bath was removed and the resulting reaction mixture was allowed to stir at room temperature overnight. The toluene layer was separated, washed with water (250 mL) and concentrated on a rotary evaporator at 85° C. under house vacuum followed by high vacuum to give the crude titled compound (225.7 g, 96.7%). ¹H NMR (CDCl₃, 400 MHz) [symbol] 4.95 (bs, 1H), 3.20 (m, 4H), 2.54 (t, 2H), 2.20 (s, 3H), 1.35 (s, 9H).

### EXAMPLE-1C

### [2-[[(4-Methyl-2,5-dioxo-4-imidazolidinyl)methyl]thio]ethyl]carbamic acid, 1,1-dimethylethyl ester

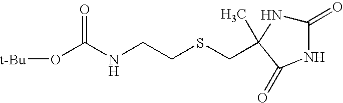

To a 3 L 4-neck RB flask equipped with an overhead stirrer, a thermocouple and a condenser connected to an empty flask and a caustic trap, was added the product of Example-1B (70 g, 0.3 mol), absolute ethanol (80 mL), sodium cyanide (19.1 g, 0.39 mol), ammonium carbonate (43.3 g, 0.45 mol) and water (720 mL) in this order. The 4^{th} neck was closed with a stopper. The resulting reaction mixture was heated at between 67 and 68° C. for 6 h. Subsequently, the almost clear brown solution was cooled to room temperature. Upon cooling, solid began to form and the heterogeneous mixture was stirred at room temperature overnight. The reaction mixture was then acidified with 12% hydrochloric acid to pH 2 in about 1 h at between −2 and 2° C. The cold reaction mixture was stirred at pH2 for additional 30 min and then filtered. The flask was rinsed with distilled water (2×250 mL) and each rinse was used to wash the solid cake. The solid was again washed with distilled water (2×250 mL) and then air-dried for 4 days. The dry solid was triturated with 200 mL of toluene for 0.5 h. The slurry was filtered. The solid was rinsed sequentially with toluene (50 mL) and 1:4 ratio of toluene/hexane (100 mL) and then air-dried at room temperature overnight to give 83.1% yield of the titled compound, m.p. 134-136° C. ¹H NMR (DMSO_{d6}, 400 MHz) δ 10.62 (s, 1H), 7.85 (s, 1H), 6.83 (m, 0.9H), 6.48 (bs, 0.1H), 3.29 (s, 2H), 2.99 (m, 2H), 2.71 (s, 2H), 2.95 (m, 2H), 1.32 (s, 9H), 1.24 (s, 3H); ¹³C NMR (DMSO_{d6}, 400 MHz), δ 178.1, 157.1, 156.1, 78.4, 63.7, 40.7, 39.4, 33.2, 28.9, 23.8. Analysis Calculated for C₁₂H₂₁N₃O₄S: C, 47.51; H, 6.98; N, 13.85; S, 10.57. Found: C, 47.76; H, 6.88; N, 13.77; S, 10.75.

### EXAMPLE-1D

### R and S-[2-[[(4-Methyl-2,5-dioxo-4-imidazolidinyl)methyl]thio]ethyl]carbamic acid, 1,1-dimethylethyl ester

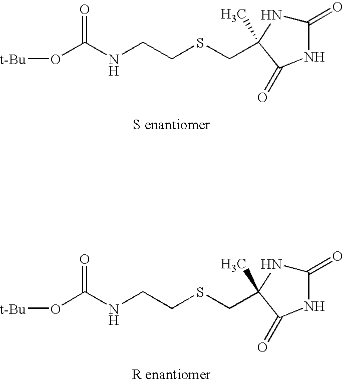

The reaction product of Example-1C was separated into its R and S enantiomers on a Chiralpak® AD column eluting with methanol. The S isomer was the first eluting isomer followed by its R enantiomer. Both isomers were used in subsequent transformations.

S enantiomer:

[α] in MeOH at 25° C.=+43.0 (365 nm). ¹HNMR: (400 mHz, CD₃OD) δ 1.49 (s, 9H), 2.05 (s, 3H), 2.65 (t, 2H), 2.9 (q, 2H, d), 3.20 (m, 2H). IR: λcm⁻¹=1772, 1709. Analysis calculated for C₁₂H₂₁N₃O₄S (formula weight=303.38): C, 47.51; H, 6.98; N, 13.85. Found: C, 47.39; H, 6.62; N, 13.83. M+H=304.

R enantiomer:

[α] in MeOH at 25° C.=−46.3 (365 nm). ¹HNMR: (400 mHz, CD₃OD) δ 1.48 (s, 9H), 2.05 (s, 3H), 2.65 (t, 2H), 2.85 (q, 2H, d), 3.18 (m, 2H). IR: λcm⁻¹=1770, 1711. Analysis calculated for C₁₂H₂₁N₃O₄S (formula weight=303.38): C, 47.51; H, 6.98; N, 13.85. Found: C, 48.15; H, 7.04; N, 14.37. M+H=304.

### EXAMPLE-1E

### S-(2-aminoethyl)-2-methyl-L-cysteine

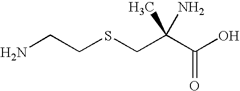

Acid Hydrolysis Method:

A 500 mL three-necked round bottom flask equipped with a distillation condenser was charged with the R-isomer product of Example-1D (45.8 g, 150.9 mmol) and treated portion wise with 48% aq. HBr (160 mL) at room temperature with stirring. After the gas evolution ceased, the mixture was heated with a heating mantle until the pot temperature reached to 126° C. while the volatile t-butyl bromide (bp 72-74° C.) followed by a small amount of aq. HBr (approx. 15 mL) were distilled off. The distillation condenser was replaced with a reflux condenser and the mixture was heated at reflux for 30 hours. The solution was concentrated and the residue was dissolved in water (250 mL) and loaded on to a Dowex® 50WX4-200 ion-exchange resin (8.5×11 cm) and eluted with water (2 L) followed by dilute aqueous ammonium hydroxide (30 mL of 28-30% ammonium hydroxide diluted to 1000 mL with water, 3 L). Fractions containing the desired product were combined, concentrated, and dried under vacuum at 75-80° C. for two hours to give 22.1 g (82%) of the title product, S-(2-aminoethyl)-2-methyl-L-cysteine, as a white solid. Proton and C-13 NMR spectra are consistent with the title product. Mp 157° C. ¹H NMR (400 MHz, D₂O) δ 1.19 (3H, s), 2.53 (1H, d, J=13.6 Hz), 2.57-2.72 (2H, m), 2.92 (1H, d, J=13.6 Hz), 2.92 (2H, t, J=6.8 Hz); ¹³C NMR (100 MHz, D₂O) δ 24.7, 31.3, 38.9, 40.9, 59.6, 180.7. Analysis Calculated for C₆H₄N₂O₂S+0.1 H₂O: C, 40.02; H, 7.95; N, 15.56; S, 17.81. Found: C, 39.93; H, 7.98; N, 15.38; S, 17.70.

Base Hydrolysis Method:

To a stainless steel autoclave equipped with agitation was added 24.2 g (0.08 moles) of the R-isomer product of Example-1D. After purging the apparatus with nitrogen, 128 g (0.32 moles) of 10% caustic was added generating a solution. The autoclave was sealed and heated to 120° C. for 30 hours. After cooling to room temperature, the autoclave was vented to give 142 ml (151 g) of an aqueous solution of the sodium salt of the title product. H¹NMR (sample acidified with HCl and diluted with D₂O, 400 MHz): δ 1.47 (s,3H), 2.75 (m, 2 H), 2.90 (d, 1H, J=14.8 Hz), 3.06 (t, 2H, J=6.4 Hz), 3.14 (d, 1H, J=14.8 Hz). C¹³NMR (sample acidified with HCl and diluted with D₂O, 100 MHz): δ 172.9, 60.8, 39.1, 39.0, 30.4, 22.2. MS (MS/CI-LC) M+1 179.

DBU (218 μL; 1.46 mmol) and ethyl acetimidate hydrochloride (171 mg; 1.34 mmol) were dissolved in ethanol (6 mL) in a 25 mL, one-necked, round-bottomed flask at room temperature (˜20° C.). The title product of Example-1E (200 mg; 1.12 mmol) was added in one portion to this solution. The mixture was stirred until the title product of Example-1E was consumed (1-2 hours). The mixture was chilled with an ice-bath and then treated with 6 M HCl (830 μL). ¹HNMR analysis indicated a chemical yield of 95 mole % or better. The solvent was evaporated and the title product of Example-1 was purified by reverse-phase or ion-exchange chromatography.

A 210 gm solution (containing ˜20 g of the title product of Example-1E of the base hydrolysis reaction product was put into a 500 mL, three-necked, round-bottomed flask. The apparatus was equipped with a mechanical stirrer, a Dean-Stark apparatus (20 mL with a stopcock), a condenser, and a temperature controller. Water (140 mL) was distilled off from the mixture. 1-butanol (150 mL) was added to the pot and the remaining water (37 mL) was distilled azeotropically. Additional 1-butanol (13 mL) was removed by distillation until the pot temperature reached 117° C. The butanol slurry was cooled to room temperature and filtered through a pad of celite. The salts were washed with 1-butanol (2×20 mL). DBU (21.8 μL; 146 mmol) and ethyl acetimidate hydrochloride (17.1 mg; 134 mmol) were dissolved in 1-butanol (40 mL) in a 500 mL, three-necked, round-bottomed flask at room temperature. The apparatus was equipped with a mechanical stirrer, an addition funnel, and a temperature probe. The title product of Example-1E/1-butanol solution was put into the addition funnel and added to the ethyl acetimidate/DBU solution while maintaining the pot temperature below 25° C. The mixture was stirred until the starting material was consumed (2-3 hours). A solution of conc. HCl (94 mL) and water (100 mL) was put into a 1 L, three-necked, round-bottomed flask and chilled to 0° C. The apparatus was equipped with a mechanical stirrer, an addition funnel, and a temperature probe. The reaction mixture was put into the addition funnel. The reaction mixture was added to the aqueous HCl solution while maintaining the temperature below 25° C. Ethyl acetate (100 mL) was added to the solution and the layers were separated. The aqueous layer was washed once more with ethyl acetate (100 mL). ¹HNMR analysis indicated a chemical yield of 95 mole % or better. This title product of Example-1 was purified by reverse-phase, ion-exchange chromatography, hydrophobic interaction chromatography, or combination thereof. ¹HNMR (400 MHz, D₂O) δ 1.49 (3H, s), 2.08 (3H, s), 2.74 (2H, m), 2.91 (1H, d), 3.17 (1H, d), 3.35 (2H, t).

### EXAMPLE 2

### Preparation of the Zwitterion

In an embodiment of the present invention, excess acid may be removed from the -[2-[(1-Iminoethyl)amino]ethyl]-2-methyl-L-cysteine dihydrochloride concentrate using anion exchange resin. It was additionally discovered that the monohydrochloro, free zwitterion, or other fractional acid derivative of S-[2-[(1-Iminoethyl)amino]ethyl]-2-methyl-L-cysteine could be prepared using the anion exchange resin. The anion exchange method is preferred for preparing the monohydrochloride and the free zwitterion due to its simplicity. S-[2-[(1-Iminoethyl)amino]ethyl]-2-methyl-L-cysteine with less than 0.5 equivalents of acid and low excess salt is especially useful for pharmaceutical preparation of alternative salt forms.

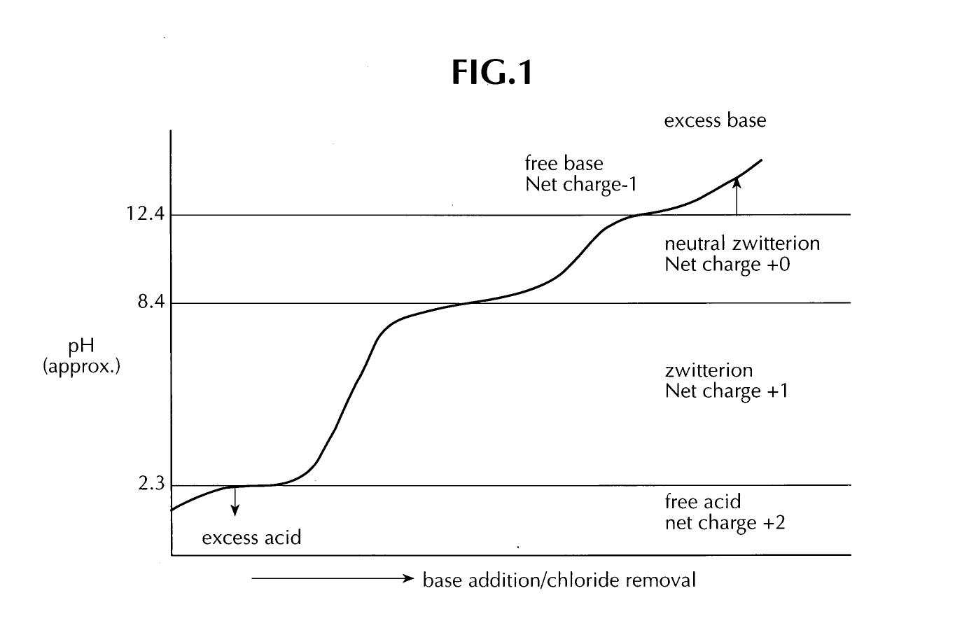

FIG. 1 shows a schematic representation of the compound titration curve. The parent S-[2-[(1-Iminoethyl)amino]ethyl]-2-methyl-L-cysteine molecule has 3 ionizable groups and can exist in 4 ionization states.

At low pH, the molecule exists as a +2 charged free acid, with the carboxylic acid, amine and amidine moieties protonated. This is the ionization state for the dihydrochloride salt.

As the pH is increased, the carboxylic acid group is the first group to deprotonate, and this produces a net charge on the molecule of +1. If the pH increase is generated by addition of sodium hydroxide to S-[2-[(1-Iminoethyl)amino]ethyl]-2-methyl-L-cysteine, the sodium dihydrochloride salt is formed. Other bases would make their corresponding salt forms. If the increase in pH is due to removal of chloride ions by anion exchange processing, the product is the monohydrochloride salt with no sodium or other counterions.

As the pH is further increased, the amine group deprotonates (about pKa=8.4) producing the neutral zwitterionic form of the molecule. A positive charge still resides on the amidine, and a negative charge still resides on the carboxyl group. In contrast, if such material is made by the addition of sodium hydroxide to the dihydrochloride, the resulting product is the monohydrochloro sodium salt, mixed with one equivalent of sodium chloride. The material prepared by the anion exchange resin approach is the free zwitterion.

Further increases in pH lead to deprotonation of the amidine ion (pKa˜12.5). The molecule in this pH range is the free base and acid salt. Note that the free base is preferably not prepared by the anion exchange method, since the negatively charged molecule binds with the anion exchange resin.

### EXAMPLE 3

### Preparation of Free Zwitterion

60 g of Amberlite IRA400 (OH) resin was prewashed with 4.7 percent (by weight) ammonium hydroxide (50 ml of 28 percent ammonium hydroxide, 250 ml deionized water), followed by extensive washing with deionized water. The final conductivity was 6.1 μS.

Samples containing about 0.9 g of S-[2-[(1-Iminoethyl)amino]ethyl]-2-methyl-L-cysteine di-hydrochloride in 142 ml HCl/water solution, were concentrated on a rotary evaporator at 60° C. to an oil. To the oil, diluted to 60 ml with deionized water, was added aliquots of 0.5 g of washed anion exchange resin while stirring. At five minutes after each aliquot of resin was added, the solution pH was measured and a sample removed through a syringe filter. A total of 9 g of anion exchange resin was added. The final pH was 10.8. The resin was removed by filtration and the filtrate was concentrated to an oil by rotary evaporation at 60° C.; no solids formed. The starting material, final filtrate and all intermediate samples were assayed by HPLC and ion chromatography for chloride.

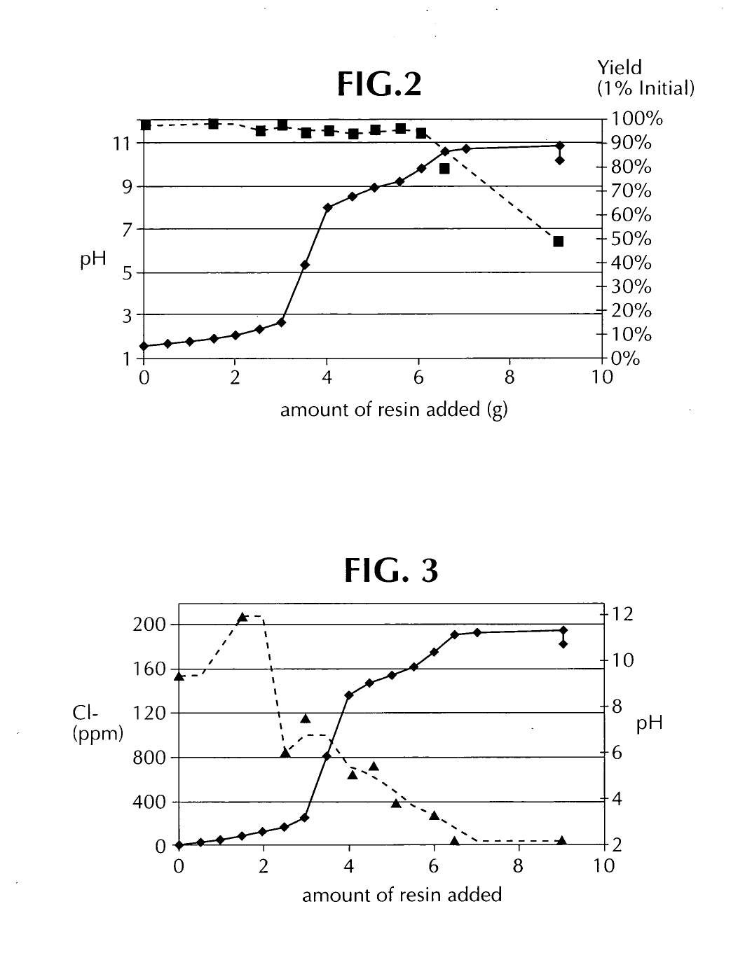

FIG. 2 shows the pseudo-titration curve for S-[2-[(1-Iminoethyl)amino]ethyl]-2-methyl-L-cysteine in water using the anion exchange resin to adjust pH. The diamond (solid line) is pH and square (dashed line) is S-[2-[(1-Iminoethyl)amino]ethyl]-2-methyl-L-cysteine (percent initial, by ion chromatography). FIG. 3 shows the pseudo-titration curve for S-[2-[(1-Iminoethyl)amino]ethyl]-2-methyl-L-cysteine in water using the anion exchange resin to adjust pH. The diamond (solid line) is pH and triangle (broken line) is chloride (by ion chromatography).

These curves are not true titration curves since samples were withdrawn during the progress of the reaction, and since true equilibrium was not attained before the increments of resin were added. Nevertheless, the graphs of FIG. 2 and FIG. 3 illustrate the expected trends. As resin is added to the S-[2-[(1-Iminoethyl)amino]ethyl]-2-methyl-L-cysteine solution the pH rises with change in slope around pH's of 2, 9 and 11. The pH's of slower rise are representative of the pK's of the carboxylic acid, amine and amidine functional groups, respectively. Above a pH of 10, the S-[2-[(1-Iminoethyl)amino]ethyl]-2-methyl-L-cysteine concentration in solution decreases. At this point, the S-[2-[(1-Iminoethyl)amino]ethyl]-2-methyl-L-cysteine is gaining a net negative charge and is binding to the resin. The chloride results show some variation between samples but in general show the trend of decreasing chloride content with increasing pH. The final chloride content is approximately 0.04 mol equivalents. HPLC assay of the samples showed no degradation.

### EXAMPLE 4

### Removal of Excess HCl to Adjust Acid Equivalents

To 3.3 g of sample containing around 305 mg/ml S-[2-[(1-Iminoethyl)amino]ethyl]-2-methyl-L-cysteine dihydrochloride and 0.23 eq excess HCl, was added 16.7 g of deioinized water. The pH was 1.04. To 14 ml of this solution, prewashed Amberlite 400 (OH) resin was added to obtain a pH of 2.5. The anion exchange treatment lightened the color of the solution from light yellow to water white. The resin was removed by filtration and the starting material and filtrate product were analyzed by chloride titration and HPLC.

Qualitative analysis of the starting material and product by HPLC found no new peaks and no increase in impurities. The results from chloride analysis by titration show that the chloride was reduced from 2.18 equivalents to around 1.14 equivalents. Although not demonstrated here, the chloride could be adjusted to the desired target by addition of HCl.

### EXAMPLE 5

### Preparation of Free Zwitterion

3.3 g of a sample containing about 1 g of S-[2-[(1-Iminoethyl)amino]ethyl]-2-methyl-L-cysteine di-hydrochloride was diluted to 20 g. Aliquots of prewashed Amberlite IRA400 (OH) resin was added to the solution and samples were periodically withdrawn through a syringe filter. Intermediate resin filtrations were performed at pH of 7.1 and 8.8 by filtering off the resin in solution and then continuing to add fresh resin to the filtrate. This was done to drive the chloride removal equilibrium and minimize product adsorption. After the final pH of 11.2 was attained, the resin was filtered off. The starting materials, intermediate samples and final filtrate were analyzed.

The resulting samples were analyzed by HPLC. No difference was seen between the HPLC traces of the starting material and product at pH of 11 within a few hours. However, some degradation peaks at around 2-3 peak area % were seen in the high pH samples after storage at room temperature for around 10 days.

### EXAMPLE 6

### Preparation of Free Zwitterion

Amberlite IRA400 (Cl) resin was rinsed with 3M HCl, water, and then 3M NaOH. Aliquots were 100 ml per 10 g of resin. This procedure was repeated 3 times in order to clean the resin and in order to generate the OH form. A final rinse with water was carried out until the conductivity of the eluting water was 2 μS. The resin was then used to titrate 40 ml of a 50 mg/ml solution of S-[2-[(1-Iminoethyl)amino]ethyl]-2-methyl-L-cysteine di-hydrochloride. The concentration is expressed in terms of zwitterion equivalents. Aliquots were taken throughout the titration, filtered and analyzed by HPLC. Subsamples of the aliquots were saved for a second HPLC analysis 1 week after the titration was performed in order to assess the stability of the samples. Additional aliquots were taken for Cl analysis using an ion selective electrode. The pH was also monitored throughout the titration.

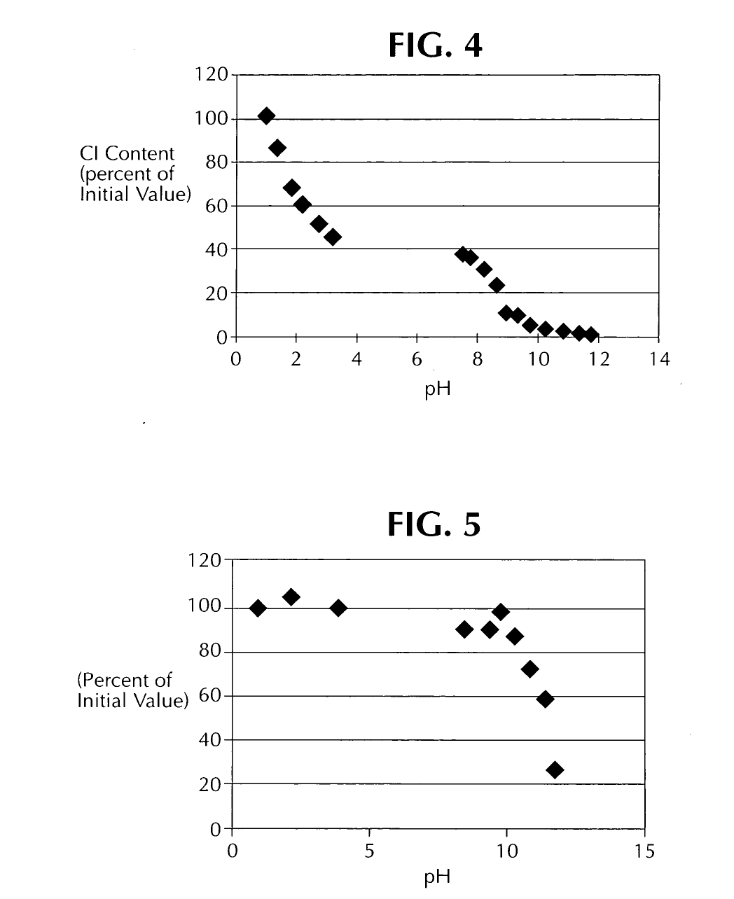

The results found in this example mirrored the results found in Example 3. The chloride specific electrode used here to measure the Cl content produced data that were much less noisy (see FIG. 4). Note that the data indicate that in removing 98% of the Cl a pH of ˜10.85 is reached. More Cl can be removed but this produced significant binding of compound to the resin (see FIG. 5). This loss of compound due to resin binding can be minimized by filtering off the resin toward the end of titration and replacing a small amount of fresh resin. This practice helps drive the equilibrium of chloride removal and minimize the sites available for compound loss by binding.

FIG. 4 Shows titration curves of S-[2-[(1-Iminoethyl)amino]ethyl]-2-methyl-L-cysteine in water with IRA-400 anion exchange resin (Rohm & Haas Amberlite, available from Rohm & Haas, Philadelphia, Pa.). FIG. 5 shows the relevant binding data associated with increasing pH.

HPLC analyses were performed using an ion pairing gradient method. The method has been shown to detect the presence of the degradation products that are expected when S-[2-[(1-Iminoethyl)amino]ethyl]-2-methyl-L-cysteine is made basic. As can be seen in the following Table 1, the data indicate that degradation is not immediate but instead occurs over a period of days.

TABLE 1
Stability of-[2-[(1-Iminoethyl)amino]ethyl]-2-methyl-L-cysteine
free zwitterion.

| | Purity At The Time | Purity One Week |
|---|---|---|
| | Of Titration | After Titration |
| Sample pH | (Peak Area %) | (Peak Area %) |
| .94 | 98.0 | 98.3 |
| 2.13 | 98.6 | 98.4 |
| 3.83 | 98.7 | 98.1 |
| 8.48 | 98.5 | 97.4 |
| 9.37 | 98.5 | 97.2 |
| 9.78 | 98.3 | 97.2 |
| 10.27 | 98.3 | 96.4 |
| 10.83 | 96.6 | 94.7 |
| 11.6 | 98.3 | 92.4 |
| 11.75 | 97.9 | 89.2 |

Samples were analyzed a few hours after preparation of the free zwitterion and again after 1 week.

HPLC Method

Pump A: 20 mM KH₂PO₄, 10 mM Pentane sulfonic acid, adjusted to pH=3 with phosphoric acid

Pump B: Acetonitrile

Gradient: 0% B at 0 min, 26% B at 15 min, 0% B at 15.1 min

Column: YMC ODS-AQ 120 A, 5 μm, 2.6×150 mm

Wavelength=205 nm

### EXAMPLE 7

### Removal of excess HCl/Preparation of Monohydrochloro 2-[(1-Iminoethyl)amino]ethyl]-2-methyl-L-cysteine

In these examples, the chloride removal process was run in batch by stirring the resin, but it could easily be run in a plant setting by recirculating the S-[2-[(1-Iminoethyl)amino]ethyl]-2-methyl-L-cysteine dihydrochloride solution over an anion exchange resin column or an anion exchange membrane. If the pH is inadvertently raised beyond the desired range, it may easily be adjusted back by adding an appropriate amount of HCl. It would be well within the ordinary skill in the art to design a large scale anion exchange process for this purpose.

### EXAMPLE 8

### Crystallization of S-[2-[(1-Iminoethyl)amino]ethyl]-2-methyl-L-cysteine maleate Form I

Zwitterion was obtained from aqueous solutions by titration with Amberlite IRA-400 resin in “OH” form. The anion exchange resin removed hydrochloride as the pH of the solution was raised to about pH 11. Filtration through a sintered glass funnel removed the resin from the solution of zwitterion. This solution was frozen with liquid nitrogen and freeze dried to obtain a glassy amorphous product. Elemental analysis of the dry glass typically indicated less than 0.3% chloride.

The initial crystallization of maleate salt was carried out with 30 mg of dry zwitterion dissolved with HPLC grade water to a final volume of 90 microliters (μl). A solution of maleic acid was made with 1.055 grams maleic acid in a 10 ml volumetric flask diluted to volume with N,N-dimethylformamide (DMF). 300 μl of maleic acid in DMF was added with stirring to the aqueous solution of zwitterion. This was two equivalent amounts of maleic acid to each equivalent of zwitterion. Acetonitrile, dried over molecular sieves, was added dropwise with stirring until the solution became turbid, then 2 to 3 drops additonal acetonitrile was added. The solution was stirred over a weekend. The resulting slurry was inspected by polarized light microscopy. Birefringent acicular crystals with positive elongation were noted. Solids were collected on a 5.0 μm Millipore LS filter and dried in vacuum at 40° C., yielding 28 mg of crystalline product. The corresponding solution with only one equivalent amount of maleic acid had gelled.

### EXAMPLE 9

### Additional Crystallization of S-[2-[(1-Iminoethyl)amino]ethyl]-2-methyl-L-cysteine maleate Form I

Subsequent small scale experiments indicated conditions for an acceptable scaleup. 1.215 grams of S-[2-[(1-Iminoethyl)amino]ethyl]-2-methyl-L-cysteine zwitterion were dissolved in about 3 ml of water. 1.283 grams of maleic acid and 12 ml of DMF were added with stirring. A clear solution was quickly obtained. 100 Ml of acetonitrile was added and the now turbid solution was seeded with crystalline S-[2-[(1-Iminoethyl)amino]ethyl]-2-methyl-L-cysteine maleate. After an hour, 13 ml additional acetonitrile was added. The slurry was stirred for another 2 hours and then the crystals were collected on several Millipore 5 μm LS filters. Because of the extremely fine particle size filtration was relatively slow. The solids were washed with a small amount of acetonitrile on the Millipore filters. These solids were transferred to a beaker and vacuum dried overnight at 40° C.

A total of five additional examples are provided to crystallize S-[2-[(1-Iminoethyl)amino]ethyl]-2-methyl-L-cysteine maleate. All the examples were conducted in the ternary solvent system (DMF/water/acetonitrile). One molar excess maleic acid was used and the ratio of DMF to water was held constant at approximately 3 to 1. Effectiveness of seeding was also evaluated in some examples

### EXAMPLE 10

### Additional Crystallization of S-[2-[(1-Iminoethyl)amino]ethyl]-2-methyl-L-cysteine maleate Form I

gram of freeze dried S-[2-[(1-Iminoethyl)amino]ethyl]-2-methyl-L-cysteine, 10 ml of DMF with 1.05 gram of dissolved maleic acid and 3.0 ml of water was added into a 125 ml jacketed reactor and stirred at 125 rpm to produce a clear solution. 100 ml of acetonitrile was intermittently added to the clear solution such that much of the turbidity created by the addition of an aliquot would subside before the addition of the next aliquot. Photo microscopy on the turbid solution suggested that the turbidity was due to liquid phase separation (emulsification) and not nucleation of crystals. The solution remained turbid upon the completion of the charge. The solution was stirred overnight. The solution had precipitated during the overnight hold, however, photo microscopy on the slurry indicated that the solids could be amorphous. S-[2-[(1-Iminoethyl)amino]ethyl]-2-methyl-L-cysteine maleate seed crystals (30 mg) were added to the slurry to promote phase conversion of the solids to crystalline S-[2-[(1-Iminoethyl)amino]ethyl]-2-methyl-L-cysteine maleate, and the system was stirred for another 24 hours. The slurry was then cooled to about −10° C. and stirred for another 24 hours to reduce product loss in the filtrate. After a total of 72 hours the slurry was filtered on a fine frit sintered glass funnel and the cake was washed with 4 ml of acetonitrile. The filtrate had a S-[2-[(1-Iminoethyl)amino]ethyl]-2-methyl-L-cysteine concentration of approximately 0.5 wt %. The solids were air dried for 1 hour and then in a vacuum oven at 50° C. for 24 hours. The solids were crystalline S-[2-[(1-Iminoethyl)amino]ethyl]-2-methyl-L-cysteine maleate by both powder x-ray defraction and DSC, however the former indicated appreciable amounts of amorphous content in the final solids.

### EXAMPLE 11

### Additional Crystallization of S-[2-[(1-Iminoethyl)amino]ethyl]-2-methyl-L-cysteine maleate Form I

Example 11 was performed at similar solvent composition and zwitterion loading, however, acetonitrile and seed addition regimes along with the hold times were rationalized to reduce amorphous content and processing time. 500 mg of S-[2-[(1-Iminoethyl)amino]ethyl]-2-methyl-L-cysteine, 502 mg of maleic acid, 5 ml of DMF and 1.5 ml of water were added to a 125 ml jacketed reactor and stirred to produce a clear solution. 31 ml of acetonitrile (63% of the total charge) was intermittently added to the solution in a manner similar to that used in Example 10. Almost all the turbidity had subsided within 1 hour of stirring after the completion of the first acetonitrile charge. The solution was then seeded with 15 mg of crystalline S-[2-[(1-Iminoethyl)amino]ethyl]-2-methyl-L-cysteine maleate crystals and stirred for 2 hours before adding the remaining 18 ml (37%) of acetonitrile. The slurry was stirred for 24 hours and then cooled to about −10° C. and stirred for another 24 hours, before being discharged onto a 150 ml fine frit sintered glass funnel. The cake was washed with 4 ml of acetonitrile, air dried for thirty minutes and then placed in a vacuum oven for 24 hours at 50° C. S-[2-[(1-Iminoethyl)amino]ethyl]-2-methyl-L-cysteine concentration in the filtrate was 0.55 wt % and the solids were crystalline S-[2-[(1-Iminoethyl)amino]ethyl]-2-methyl-L-cysteine maleate by PXRD with a halo in the baseline indicating presence of some amorphous S-[2-[(1-Iminoethyl)amino]ethyl]-2-methyl-L-cysteine maleate. The halo was however, not as pronounced as for the product from Example 10.

### EXAMPLE 12

### Additional Crystallization of S-[2-[(1-Iminoethyl)amino]ethyl]-2-methyl-L-cysteine maleate Form I

The objective of Example 12 was to explore the possibility of improving throughput of the process by decreasing the amount of acetonitrile. 0.9 grams of S-[2-[(1-Iminoethyl)amino]ethyl]-2-methyl-L-cysteine, 0.93 grams of maleic acid, 9 ml of DMF and 2.7 ml of water were added to a 125 ml jacketed reactor and stirred to affect a clear a solution. 56 ml of acetonitrile were intermittently added to the solution in a manner similar to that used in the other examples. 23 mg of S-[2-[(1-Iminoethyl)amino]ethyl]-2-methyl-L-cysteine maleate seed crystals were added once all the turbidity had subsided after the completion of acetonitrile charge. The system was stirred for 24 hours at 175 rpm and then the slurry was discharged on a 150 ml fine frit sintered glass funnel. The cake was washed with 5 ml of acetonitrile; air dried for forty minutes and then placed in a vacuum oven at 50° C. for 24 hours. Approximately 600 mg of product was recovered after drying. Concentration of S-[2-[(1-Iminoethyl)amino]ethyl]-2-methyl-L-cysteine in the filtrate was approximately 1.0 wt %. PXRD analysis on the product indicated that it had very little amorphous content. Solution NMR showed that the product could have as much as 0.1 molar equivalent of trapped DMF. Comparison of solids in the slurry prior to filtration and after oven drying through photomicroscopy show significant morphological differences.

### EXAMPLE 13

### Additional Crystallization of S-[2-[(1-Iminoethyl)amino]ethyl]-2-methyl-L-cysteine maleate Form I

Example 13 was performed to explore if seeding could be eliminated as an aide to induce nucleation. The remainder of the procedure for this experiment was identical to that for Example 12. Filtrate concentration and solid-state attributes on the product indicated that comparable performance could be obtained without seeding.

### EXAMPLE 14

### Additional Crystallization of S-[2-[(1-Iminoethyl)amino]ethyl]-2-methyl-L-cysteine maleate Form I

Example 14 was conducted to explore the possibility of improving yield of the procedure used in experiments 3 and 4 by cooling and adding more acetonitrile once a significant fraction of solids had already precipitated. The procedure involved adding 1.0 grams of S-[2-[(1-Iminoethyl)amino]ethyl]-2-methyl-L-cysteine, 1.03 grams of maleic acid, 10 ml DMF and 3.0 ml of water to a 125 ml jacketed reactor and stirring to produce a clear solution. 56 ml of acetonitrile were intermittently added to the solution in a manner similar to that used in the above examples. 28 mg of S-[2-[(1-Iminoethyl)amino]ethyl]-2-methyl-L-cysteine maleate seed crystals were added after substantially all the turbidity had subsided subsequent to the completion of acetonitrile charge. Significant precipitation had occurred within the first 2 hours after seeding and the slurry was cooled to about −10° C. on the jacket. The slurry was stirred at this temperature for 24 hours.

Supernatant analysis over the cool down and hold period indicated only marginal reduction in concentration and therefore 25 ml of acetonitrile was added to determine if anti-solvent addition would improve yield. After the addition of more acetonitrile the slurry was brought back to 25° C. and stirred for another 24 hours before being discharged on a 150 ml fine frit sintered glass funnel. The cake was washed with 5 ml of acetonitrile.

Unlike any cake previously observed, the cake from this example was sticky and very wet even after 2 hours of air-drying on the filter. These solids were placed in a vacuum oven without heat for 4 hours to remove loose solvent. Drying at 50° C. in the vacuum oven for 24 hours followed. Concentration of the filtrate did not identify any significant advantage of adding more anti-solvent. PXRD analysis on the solids from this example immediately after drying did show some amorphous solids mixed in with S-[2-[(1-Iminoethyl)amino]ethyl]-2-methyl-L-cysteine maleate crystals.

### EXAMPLE 15

### Crystallization Procedures for Form II

A ternary solvent system comprising of water, dimethyl formamide (DMF) and acetonitrile (ACN) was determined as the most effective solvent system for the crystallization of maleate salts of S-[2-[(1-Iminoethyl)amino]ethyl]-2-methyl-L-cysteine.

A new variant of the original ternary solvent system was identified and developed in the process of discovering form II. This procedure was further refined to improve operability by eliminating the need to seed for inducing nucleation. The description of the routes to make form II is as follows.

In the first procedure, 200 milligrams of S-[2-[(1-Iminoethyl)amino]ethyl]-2-methyl-L-cysteine and 206 milligrams of maleic acid were dissolved in a mixture of 0.86 ml of DMF and 0.3 ml of water. Once a clear solution had been obtained after agitation through a magnetic bead, 5 ml of ACN were intermittently added under agitation. Adequate time was afforded for turbidity to subside after the addition of each aliquot of ˜0.5 ml of ACN. A clear solution was obtained after the completion of ACN charge. 10 milligrams of form I seeds were added to induce nucleation in the solution. The slurry was stirred at room temperature for 24 hrs. It was filtered on a 30 ml fine frit sintered glass funnel. The cake was washed with 2 ml of ACN and then air dried for 10 minutes. A portion of the air dried solids was dried in a vacuum oven at 60° C. under 28 inches Hg vacuum. Though the air-dried solids de-hydrated during oven drying, they re-hydrated to form II upon removal from the oven in less than 30 minutes. The fact that form I (used to induce crystallization) was not detected in the isolated product, indicated that under these crystallization conditions form II could be more stable than form I. Yield for this procedure was estimated to be approximately 65% on weight basis. Induction time for primary nucleation was approximately 30 minutes at sub-gram scale.

### Characterization of S-[2-[(1-Iminoethyl)amino]ethyl]-2-methyl-L-cysteine maleate Form I

Birefringent, acicular crystals with positive elongation by polarized light microscopy were observed in the slurry before collection by filtration and in the isolated product after vacuum drying at 40° C.

S-[2-[(1-Iminoethyl)amino]ethyl]-2-methyl-L-cysteine maleate crystalline salt Form I absorbs less than one percent water by weight at 90% R.H. and 25° C., and melts at 123° C. S-[2-[(1-Iminoethyl)amino]ethyl]-2-methyl-L-cysteine maleate crystalline salt Form I has an aqueous solubility in excess of 230 mg ml⁻¹.

Table 2 shows the elemental analysis of S-[2-[(1-Iminoethyl)amino]ethyl]-2-methyl-L-cysteine maleate crystalline salt Form I, as well as theoretical composition at 1.5% water content.

TABLE 2
Elemental Analysis of
S-[2-[(1-Iminoethyl)amino]ethyl]-2-methyl-L-cysteine
maleate crystalline salt Form I, Measured by combustion
analysis vs. theory; weight percent

| | | | Theory |
|---|---|---|---|
| | | | 1:1 at 1.5% |
| | Example 9 | Example 8 | H2O |
| Carbon | 42.30 | 42.37/42.37 | 42.3 |
| Hydrogen | 6.50 | 6.52/6.56 | 6.4 |
| Nitrogen | 12.37 | 12.36/12.31 | 12.3 |
| Sulfur | 8.64 | 9.21/9.16 | 9.4 |
| Water* | 1.5% | 1.5% | |
| Melting point | 123° C. dsc | 123° C. dsc, | |
| | | hotstage | |
| H-NMR | 1:1 consistent | 1:1 consistent | |
| DMF (NMR) | 0.16 equivalent | 0.2 equivalent | |

*Karl Fischer coulometric water analysis.

Proton NMR was consistent with the structure and stoichiometry of a 1:1 combination of S-[2-[(1-Iminoethyl)amino]ethyl]-2-methyl-L-cysteine zwitterion and maleic acid. Proton NMR also showed 0.2 equivalents (3 to 3.5 percent) DMF in Example 8, which does not significantly alter the expected elemental analysis, within ±0.4.

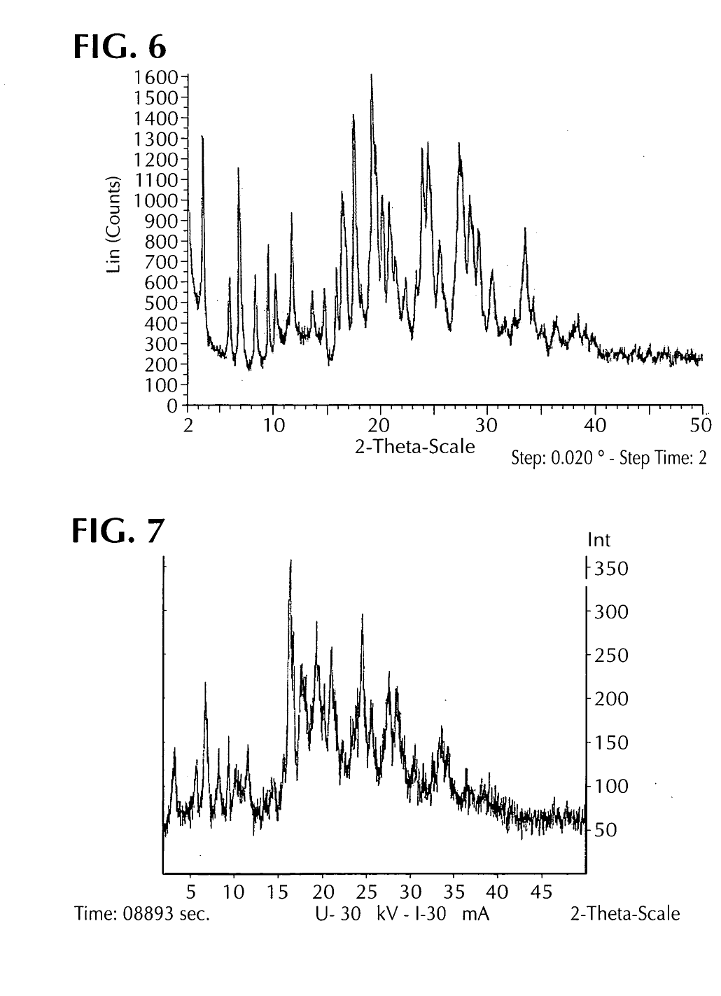

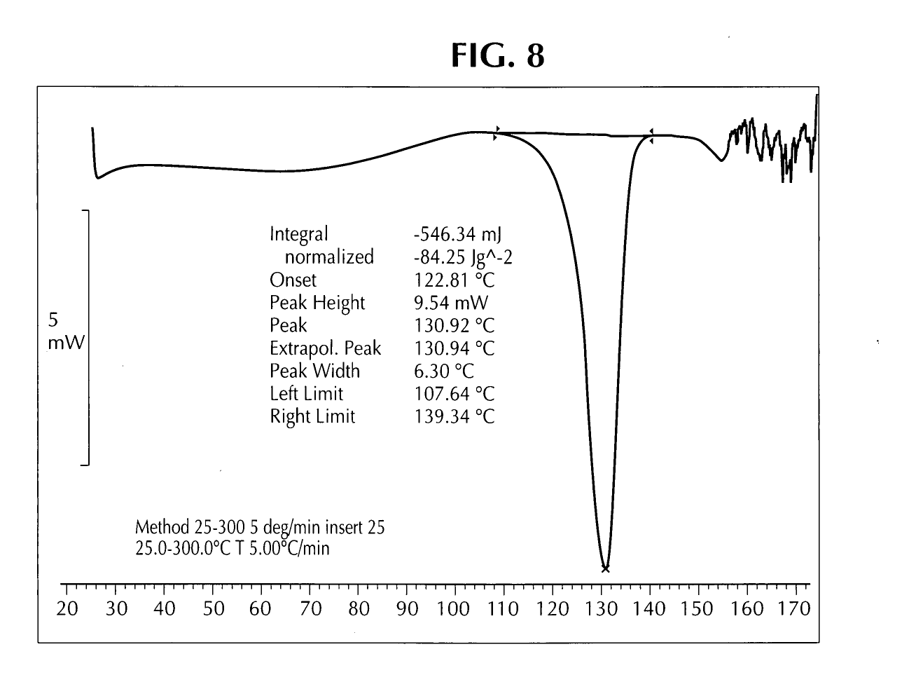

FIG. 6 is a powder x-ray pattern of a sample (Example 9) of S-[2-[(1-Iminoethyl)amino]ethyl]-2-methyl-L-cysteine maleate. FIG. 7 is a powder x-ray pattern of a sample (Example 8) of S-[2-[(1-Iminoethyl)amino]ethyl]-2-methyl-L-cysteine maleate. Both Example 8 and Example 9 show characteristic peaks useful in characterizing crystalline S-[2-[(1-Iminoethyl)amino]ethyl]-2-methyl-L-cysteine maleate.

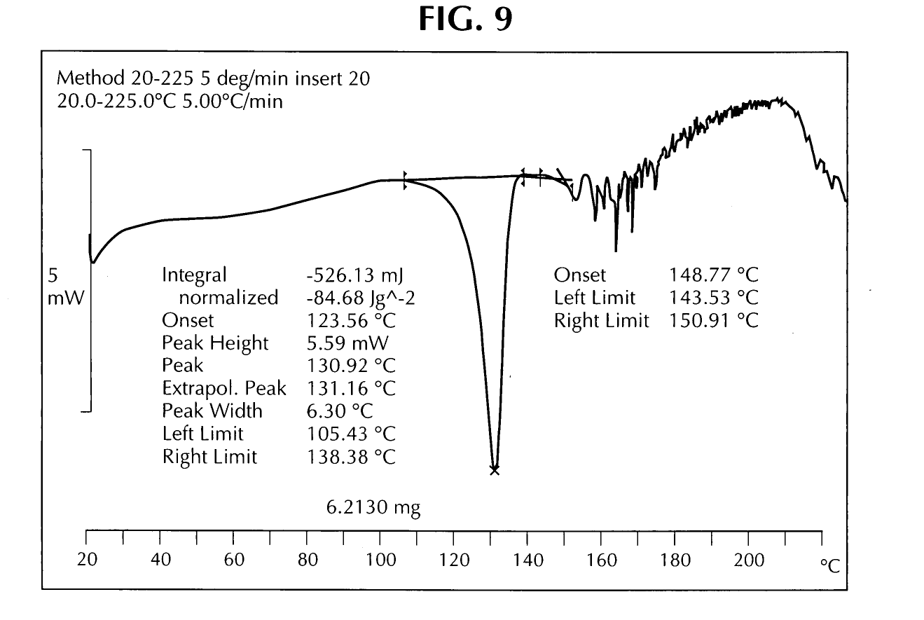

FIG. 8 is a graph of a differential scanning calorimetry study of a sample (10.046 mg. Example 9) of S-[2-[(1-Iminoethyl)amino]ethyl]-2-methyl-L-cysteine maleate. FIG. 9 is a graph of a differential scanning calorimetry study of a sample (6.2130 mg. Example 8) of S-[2-[(1-Iminoethyl)amino]ethyl]-2-methyl-L-cysteine maleate;

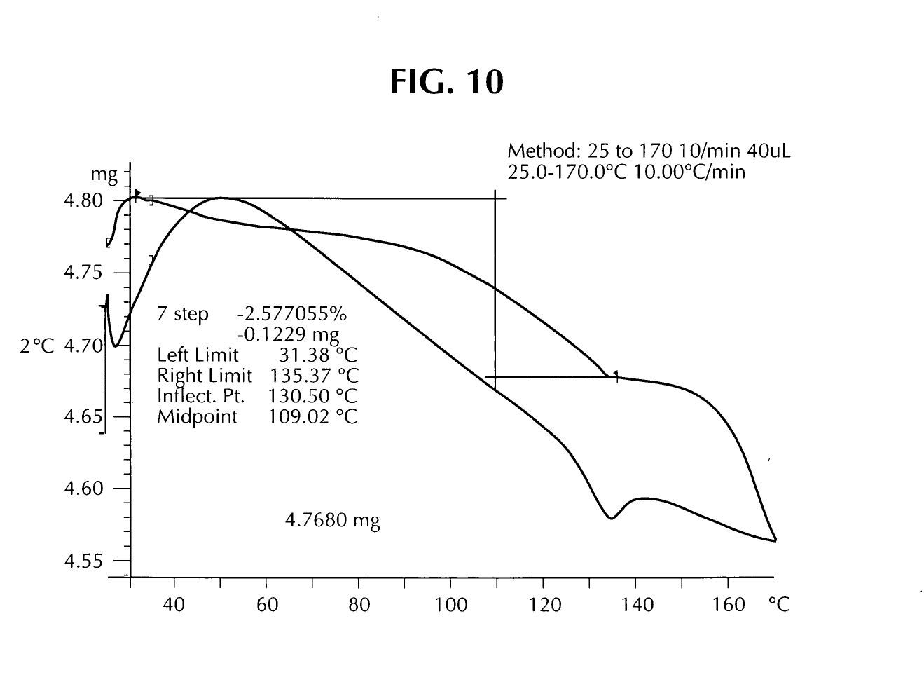

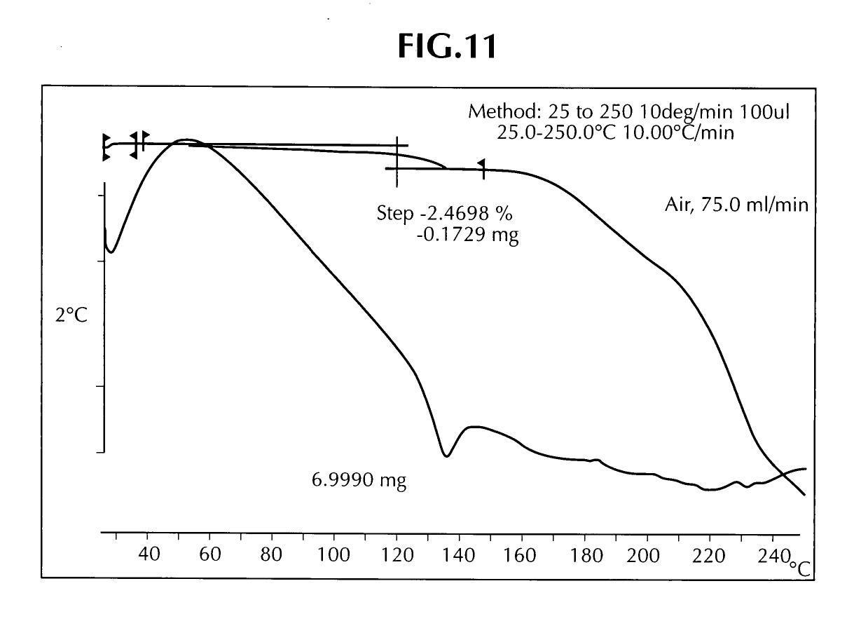

FIG. 10 is a thermogravimetric plot of a sample (4.7680 mg. Example 9) of S-[2-[(1-Iminoethyl)amino]ethyl]-2-methyl-L-cysteine maleate. FIG. 11 is a thermogravimetric plot of a sample (Example 8) of S-[2-[(1-Iminoethyl)amino]ethyl]-2-methyl-L-cysteine maleate.

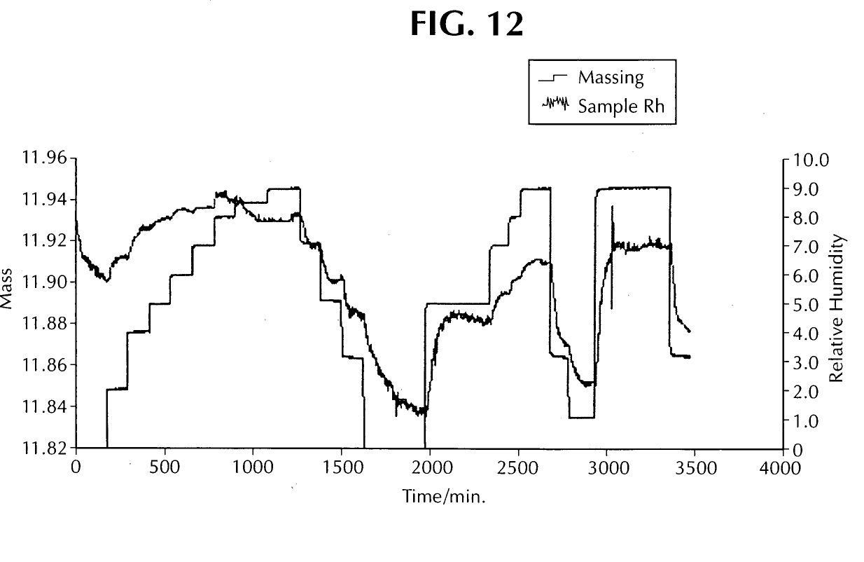

FIG. 12 is a plot of a moisture sorption study of S-[2-[(1-Iminoethyl)amino]ethyl]-2-methyl-L-cysteine maleate.

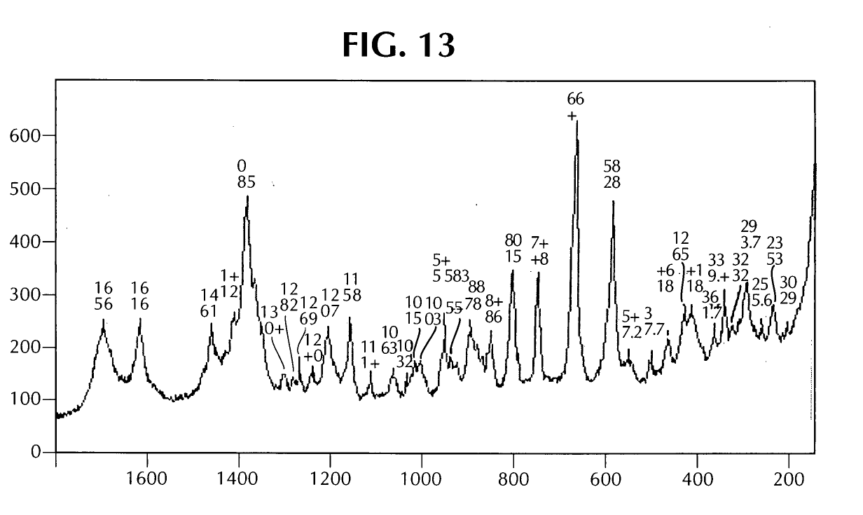

FIG. 13 shows the Raman spectrum of the crystalline S-[2-[(1-Iminoethyl)amino]ethyl]-2-methyl-L-cysteine maleate. Briefly, the Raman spectrum is a vibrational signature of a molecule or complex system. Its origin lies in the inelastic collisions between the molecules and photons, which are the particles of light composing a light beam. The collision between the molecules and the photons leads to an exchange of energy with consequent change in energy and hence wavelength of the photon. Thus, a Raman spectrum is a set of very narrow spectral lines emitted from object molecules when illuminated by an incident light. The width of each spectral line is strongly affected by the spectral width of the incident light and hence tightly monochromatic light sources, such as lasers, are used. The wavelength of each Raman line is expressed as a wave number-shift from the incident light, which is the difference between the inverse wavelength of the Raman line and the incident light. The wave number-shift, not the absolute wavelength, of the Raman lines is specific to particular atomic groups in molecules. Raman spectra measure the vibration states of molecules, which are determined by their molecular structure.

### Characterization of S-[2-[(1-Iminoethyl)amino]ethyl]-2-methyl-L-cysteine maleate Form II

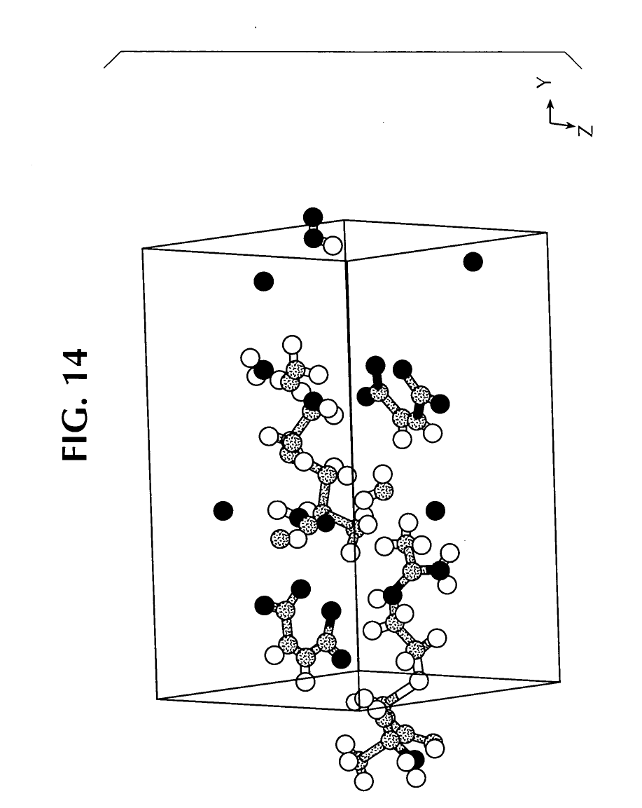

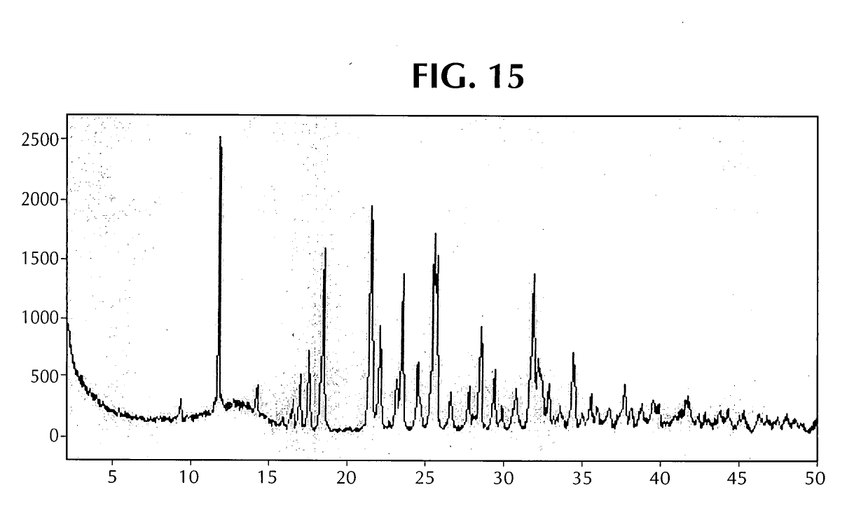

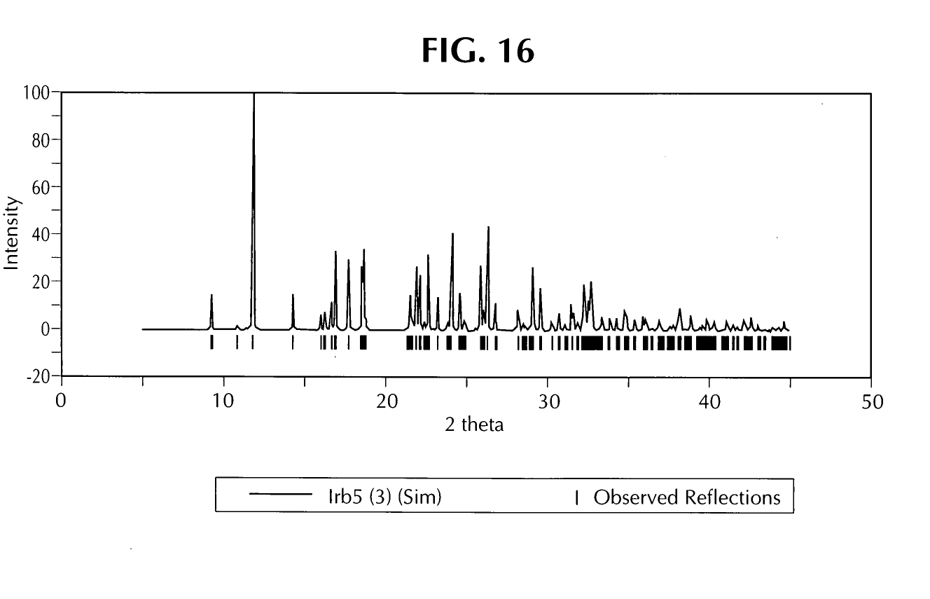

Several large crystals were isolated from phase stability studies performed by slurrying forms I and II mixtures at 5° C. in 70/30 v/v ACN/water solvent mixture over three weeks. The stoichiometry of the unit cell was determined to be: two and half molecules of water for each molecule to S-[2-[(1-Iminoethyl)amino]ethyl]-2-methyl-L-cysteine and maleic acid. The crystalline structure has been solved by single crystal x-ray diffraction. FIG. 14 shows a unit cell of S-[2-[(1-Iminoethyl)amino]ethyl]-2-methyl-L-cysteine maleate Form II. The space group was P₂₁ (monoclinic) and the unit cell parameters were; a=8.7002, b=19.0009, c=8.5562° A, α=90, β=34.439, γ=90°. The structure was that of a channel hydrate, with water molecules located in channels running along the short c axis. The calculated x-ray powder pattern from the single crystal structure on S-[2-[(1-Iminoethyl)amino]ethyl]-2-methyl-L-cysteine maleate compared very well with the observed pattern for form II (See FIG. 15 and FIG. 16).

The solid S-[2-[(1-Iminoethyl)amino]ethyl]-2-methyl-L-cysteine maleate Form II was highly crystalline by polarized light microscopy and the crystallite size was on the order of twenty micrometers. The particles were bar-like. Elemental analysis of S-[2-[(1-Iminoethyl)amino]ethyl]-2-methyl-L-cysteine maleate Form II performed by physical methodology provided a very tight correspondence with the theory for a hydrate with 2.5 moles of water per mole of S-[2-[(1-Iminoethyl)amino]ethyl]-2-methyl-L-cysteine maleate (see Table II). Karl Fischer water analysis found water at 10.97% compared to a theoretical value of 11.84% for hemi-penta-hydrate.

Solubility of S-[2-[(1-Iminoethyl)amino]ethyl]-2-methyl-L-cysteine maleate Form II crystalline salt was measured in 70/30 and 90/10 v/v acetonitrile/ethanol 3A mixtures at 5 and 25° C. Excess solids were equilibrated in these solvents at the appropriate temperature (in a shaker bath) for nineteen days. Supernatant samples were withdrawn and analyzed by HPLC for concentration of S-[2-[(1-Iminoethyl)amino]ethyl]-2-methyl-L-cysteine. Table 4 summarizes the result. While the solubility decreases significantly with the presence of more acetonitrile in the solvent system, the effect with temperature is almost negligible in the 20° range.

TABLE 3
Solubility of Form II in Different Proportions
of acetonitrile/ethanol 3A

| | 70/30 ACN/EtOH 3A | 90/10 ACN/EtOH 3A |
|---|---|---|
| | wt. % S-[2-[(1- | wt. % S-[2-[(1- |
| | Iminoethyl)amino]ethyl]- | Iminoethyl)amino]ethyl]-2- |
| Temperature ° C. | 2-methyl-L-cysteine | methyl-L-cysteine |
| 5 | 0.679 | 0.169 |
| 25 | 0.737 | 0.135 |

Attempts were made to study the solubility of S-[2-[(1-Iminoethyl)amino]ethyl]-2-methyl-L-cysteine maleate Form II crystalline salt at 5° C. in 70/30 v/v acetonitrile/water with and without excess (1 molar equivalent) maleic acid and in 90/10 v/v acetonitrile/water with one molar equivalent excess maleic acid All three experimental vials were prepared by adding approximately 90 mg of S-[2-[(1-Iminoethyl)amino]ethyl]-2-methyl-L-cysteine maleate crystalline salt and 30 mg of maleic acid to approximately 300 mg of binary solvent. These vials were then placed in a shaker bath at 5° C. After four days of equilibration when the vials were observed, it was discovered that the system had turned from a suspension of solids in a single liquid phase to a biphasic liquid with no suspended solids. Both layers from each of the three vials were analyzed for water content (through Karl-Fischer analysis) and concentration of the compound. Table 4 summarizes the results. Only the compound concentrations in the acetonitrile rich (top) layer could represent true solubility of S-[2-[(1-Iminoethyl)amino]ethyl]-2-methyl-L-cysteine maleate salt in a solvent system with the composition calculated based on KF analysis. It is worth pointing out though that the exact conditions leading to measured concentrations in the top layer could only be truly established when the partitioning of excess maleic acid is established between the two layers. The bottom layer (rich in water) would most likely be under-saturated since no excess solids remained. This possibility is substantiated by the solubility data on the bottom layer, where the concentrations are found to be very similar across all the experiments. While the vial at 70/30 acetonitrile/water with excess maleic acid remained biphasic even after nineteen days, the 70/30 acetonitrile/water vial without any excess maleic acid after five days had some very large crystals floating in a solution that still seemed biphasic. This experiment was terminated to isolate and characterize the solids, which were found to be form II. These crystals were used to obtain crystallography data. After nineteen days of equilibration at 5° C., the 90/10 acetonitrile/water vial with excess maleic acid had turned back into a slurry of solids in a single liquid phase. The solids were isolated and found to be form II and the concentration in the solution phase was measured to be 0.613 wt % S-[2-[(1-Iminoethyl)amino]ethyl]-2-methyl-L-cysteine. The fact that 70/30 vial with excess maleic acid remained biphasic and more importantly without any re-crystallized solids could be because of the higher solubility resulting from excess maleic acid (compared to 70/30 vial without excess malice acid) and higher water content (compared to the 90/10 vial with excess maleic acid). Equally interesting to note is that the two vials where crystallization did occur from biphasic solvent systems had form II crystals in them at the time of isolation. This observation would seem in-line with the expectations from the water-activity hydration state phase diagram assuming that the excess maleic acid does not have any bearing on the solid form stability.

TABLE 4
Concentration and KF data from solubility experiments in ACN/water

| | 70/30 ACN/Water | | 70/30 ACN/Water | | 90/10 ACN/Water | |
|---|---|---|---|---|---|---|
| | (1x excess maleic acid) | | (no excess maleic acid) | | (1x excess maleic acid) | |
| | KF | Concentration Wt % | KF | Concentration Wt % | KF | Concentration Wt % |
| Top Layer | 21.0 | 2.71 | 18.57 | 1.02 | 9.07 | 2.67 |
| Bottom | 30.27 | 5.40 | 46.65 | 6.68 | 26.63 | 5.28 |
| Layer | | | | | | |

The final set of solubility studies were conducted in ternary solvent systems of dimethyl formamide (DMF)/water/acetonitrile (ACN). This system was studied because most of the early success in crystallizing S-[2-[(1-Iminoethyl)amino]ethyl]-2-methyl-L-cysteine maleate salt was found in this solvent mixture. All the experiments used one molar equivalent excess maleic acid and the solvent compositions selected were 15/15/70, 5/5/90, 20/10/70, 6.6/3.4/90, 22.5/7.5/70, 7.5/2.5/90 v/v DMF/water/ACN. Experiments were only conducted for the last four of these compositions at 25° C., but the data was collected for the compositions at 5° C. Supernatant was analyzed after four and nineteen days. Equilibrated solids were also isolated and characterized after nineteen days from most experiments (see Table 5). All the experiments with 70% ACN in the solvent system where solids were isolated led to the complete conversion of form I to form II during the nineteen days, while a few experiments with 90% ACN in the solvent system retained the original form I as the solid state after the equilibration period. The latter could be because of the further reduction in water activity of these compositions due to the addition of DMF, pushing the system towards conditions where form I is more stable. Solid-state data from 7.5/2.5/90 DMF/water/ACN system however, defies the trend and is very difficult to explain with the limited information available. Table 5, lists concentration data in wt % S-[2-[(1-Iminoethyl)amino]ethyl]-2-methyl-L-cysteine from all the experiments at 5 and 25° C. after four and nineteen days of equilibration. The most obvious trend is that the solubility greatly increases when the ACN content of the system is reduced from 90% to 70%. There is also some temperature dependence for solubility across all the experiments. When the data is analyzed at a fixed ACN content, it seems that the solubility initially decreases before increasing as the DMF content of the system is increased at the expense of water. This trend seems to hold for both 70 and 90% ACN. It is worth stressing that while the complete phase transformation to the most stable form might have occurred in all these experiments, the solubility values reported in table 5 may not be the equilibrium, which is always slow to achieve from super-saturation (as would be the case for all the experiments where transformation to form II occurred). In summary, though this solvent system is complex, it does offer regions of solvent composition conducive to crystallization process design especially for form II.

TABLE 5
Solubility data in DMF/water/acetonitrile ternary solvent system

| | 15/15/70 | | 5/5/90 | | 20/10/70 | | 6.3/3.4/90 | | 22.5/7.5/70 | | 7.5/2.5/90 | |
|---|---|---|---|---|---|---|---|---|---|---|---|---|
| | D/W/A | | D/W/A | | D/W/A | | D/W/A | | D/W/A | | D/W/A | |
| | 5° C. | 25° C. | 5° C. | 25° C. | 5° C. | 25° C. | 5° C. | 25° C. | 5° C. | 25° C. | 5° C. | 25° C. |
| | Wt % | Wt % | Wt % | Wt % | Wt % | Wt % | Wt % | Wt % | Wt % | Wt % | Wt % | Wt % |
| 4 Days | 3.31 | N/A | 1.15 | N/A | 2.91 | 3.46 | 1.35 | 2.13 | 3.06 | 4.90 | 4.19 | 2.53 |
| 19 Days | 5.71 | N/A | 1.16 | N/A | 1.75 | 2.33 | 1.02 | 1.74 | 2.75 | 7.38 | 2.12 | 2.45 |
| Form | N/A | N/A | I | N/A | II | N/A | I | I | II | II | II | II |
| 19 Days | | | | | | | | | | | | |

TABLE 6
Elemental analysis results
Measured by combustion analysis vs. theory; weight percent.

| Element | Measured | Theory 2.5 H₂O |
|---|---|---|
| Carbon | 37.90 | 37.89 |
| Hydrogen | 6.55 | 6.89 |
| Nitrogen | 11.06 | 11.05 |
| Sulphur | 8.78 | 8.43 |

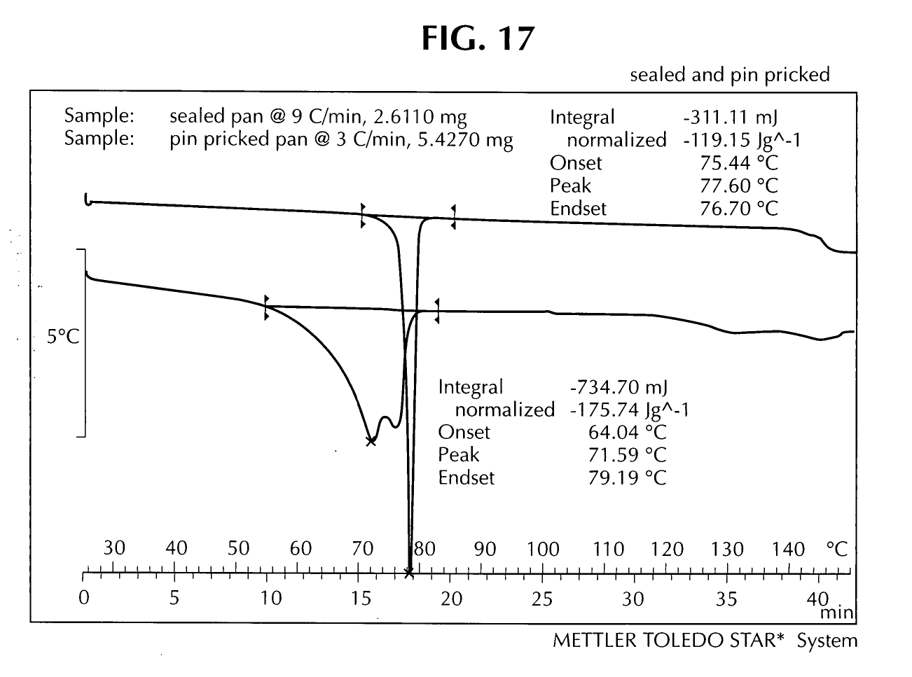

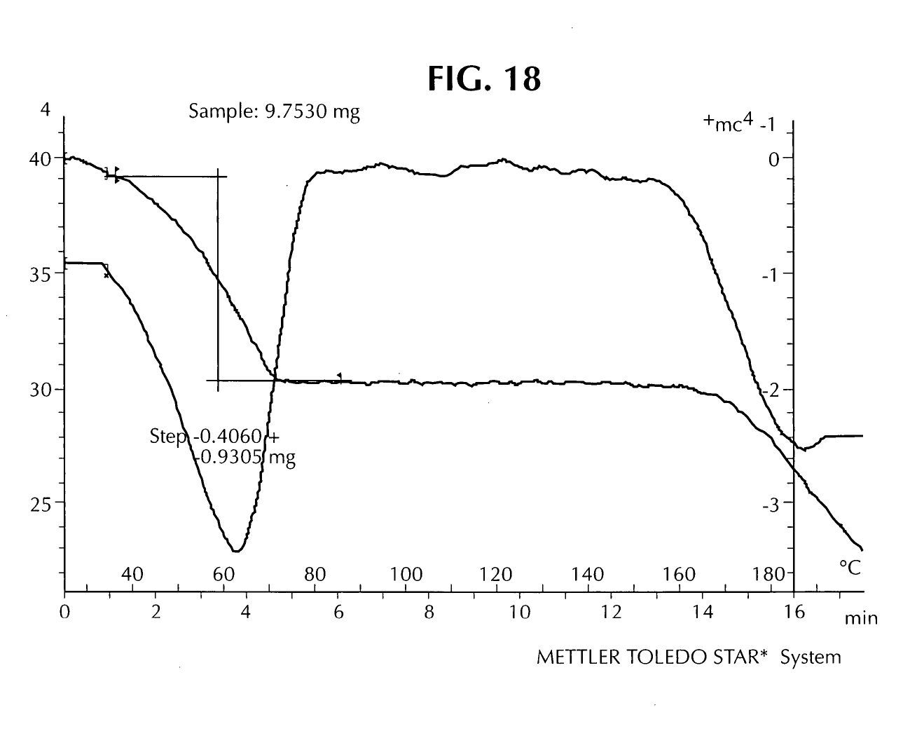

Referring to FIG. 17, differential scanning calorimetry (DSC) found a single eutectic melt (with water) at 77.69° C. Thermogravimetric analysis (TGA) showed a weight loss of 8.8% between 45 and 80° C. TG-IR indicated that all the weight loss was due to water loss (see FIG. 18). Whilst 8.8% loss is lower than the value for water measured by KF, it is reasonably close for a hydrate of this nature.

Proton NMR analysis was conducted on S-[2-[(1-Iminoethyl)amino]ethyl]-2-methyl-L-cysteine maleate (form II) to determine if this form trapped any DMF during the crystallization process. The data showed that there was no detectable DMF in the crystals.

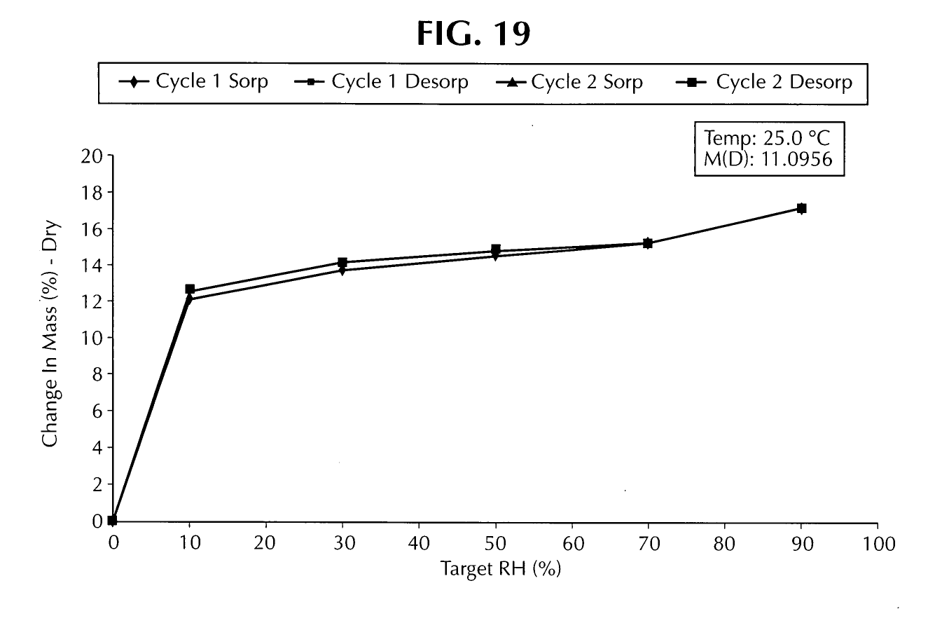

Referring to FIG. 19, moisture sorption of S-[2-[(1-Iminoethyl)amino]ethyl]-2-methyl-L-cysteine maleate (form II) at 25° C., by DVS moisture balance, showed that the hydrate could be de-hydrated relatively easily by lowering relative humidity (R.H.) to 0%. However, the solids re-hydrate equally easily when exposed to R.H. greater than 10%. The experiment showed a moisture gain of ˜2.5% (0.5 mole of water) between 10 and 70% R.H. Between 70 and 90% R.H. the gain was ˜4.5% (1 mole of water). Cycling between sorption and de-sorption cycles did not show any hysteresis or loss of crystallinity. The behavior of form II during this experiment was typical of a highly crystalline channel hydrate/solvate.

Pharmaceutical Compositions

Also embraced within this invention is a class of pharmaceutical compositions comprising crystalline S-[2-[(1-Iminoethyl)amino]ethyl]-2-methyl-L-cysteine maleate Form II in association with one or more non-toxic, pharmaceutically-acceptable carriers and/or diluents and/or adjuvants (collectively referred to herein as “carrier” materials) and, if desired, other active ingredients. The crystalline Form II of S-[2-[(1-Iminoethyl)amino]ethyl]-2-methyl-L-cysteine maleate of the present invention may be administered by any suitable route, preferably in the form of a pharmaceutical composition adapted to such a route, and in a dose effective for the treatment intended. The active S-[2-[(1-Iminoethyl)amino]ethyl]-2-methyl-L-cysteine maleate Form II and compositions may, for example, be administered orally, intravascularly, intraperitoneally, subcutaneously, intramuscularly or topically.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are tablets or capsules. The active ingredient may also be administered by injection as a composition wherein, for example, saline, dextrose or water may be used as a suitable carrier.

The amount of therapeutically active compound that is administered and the dosage regimen for treating a disease condition with the compound and/or compositions of this invention depends on a variety of factors, including the age, weight, sex and medical condition of the subject, the severity of the disease, the route and frequency of administration, and the particular compound employed, and thus may vary widely. The pharmaceutical compositions may contain active ingredients in the range of about 0.1 to 2000 mg, preferably in the range of about 0.5 to 500 mg and most preferably between about 1 and 100 mg. A daily dose of about 0.01 to 100 mg/kg body weight, preferably between about 0.5 and about 20 mg/kg body weight and most preferably between about 0.1 to 10 mg/kg body weight, may be appropriate. The daily dose can be administered in one to four doses per day.

Crystalline S-[2-[(1-Iminoethyl)amino]ethyl]-2-methyl-L-cysteine maleate Form II can also be administered by a transdermal device. Preferably topical administration will be accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent may also function as the membrane.

The oily phase of the emulsions of this invention may be constituted from known ingredients in a known manner. While the phase may comprise merely an emulsifier, it may comprise a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. Emulsifiers and emulsion stabilizers suitable for use in the formulation of the present invention include Tween 60, Span 80, cetostearyl alcohol, myristyl alcohol, glyceryl monostearate, and sodium lauryl sulfate, among others.

The choice of suitable oils or fats for the formulation is based on achieving the desired cosmetic properties, since the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters may be used. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredients are dissolved or suspended in suitable carrier, especially an aqueous solvent for the active ingredients. The active ingredients are preferably present in such formulations in a concentration of 0-5 to 20%, advantageously 0.5 to 10% and particularly about 1.5% w/w.

For therapeutic purposes, S-[2-[(1-Iminoethyl)amino]ethyl]-2-methyl-L-cysteine maleate is ordinarily combined with one or more adjuvants appropriate to the indicated route of administration. If administered per os, the compound may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The crystalline S-[2-[(1-Iminoethyl)amino]ethyl]-2-methyl-L-cysteine maleate may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

The invention being thus described, it is apparent that the same can be varied in many ways. Such variations are not to be regarded as a departure from the spirit and scope of the present invention, and all such modifications and equivalents as would be obvious to one skilled in the art are intended to be included within the scope of the following claims.

What is claimed is:

1. A crystalline form of S-[2-[(1-Iminoethyl)amino]ethyl]-2-methyl-L-cysteine maleate characterized by at least one of: melting point of about 77.69° C.; x-ray powder pattern substantially as shown in FIG. 15; x-ray powder pattern substantially as shown in FIG. 16; unit cell as shown in FIG. 14; and elemental analysis substantially as in Table 7.

2. A pharmaceutical composition comprising the crystalline S-[2-[(1-Iminoethyl)amino]ethyl]-2-methyl-L-cysteine maleate Form II of claim 1 together with a pharmaceutically acceptable carrier.

3. A method of treating a condition wherein pathologically high production forms a part in a subject in need of such treatment comprising administering to the subject an effective amount of the crystalline S-[2-[(1-Iminoethyl)amino]ethyl]-2-methyl-L-cysteine maleate Form II of claim 1.

4. A method of decreasing nitric oxide production in a subject comprising administering to the subject an effective amount of the crystalline S-[2-[(1-Iminoethyl)amino]ethyl]-2-methyl-L-cysteine maleate Form II of claim 1.
